(19) United States
(12) Patent Application Publication
(10) Pub. No.: US 2010/0179093 A1
(43) Pub. Date: Jul. 15, 2010

(54) Neoropeptide-2-Receptor (Y-2R) Agonists

(76) Inventors: Waleed Danho, Wayne, NJ (US); Nader Fotouhi, Basking Ridge, NJ (US); David Charles Fry, Langhorne, PA (US); Wajiha Khan, East Hanover, NJ (US); Joseph Swistok, Nutley, NJ (US); Jefferson Wright Tilley, North Caldwell, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/610,417

(22) Filed: Nov. 2, 2009

Related U.S. Application Data
(60) Provisional application No. 61/111,442, filed on Nov. 5, 2008.

(51) Int. Cl. A61K 38/16; C07K 14/00; C07K 7/00; A61K 38/10; A61P 3/04; A61P 3/10

(52) U.S. Cl. 514/12; 530/324; 530/326; 514/13

(57) ABSTRACT

Provided herein are neuropeptide-2 receptor agonists of the formula (I):

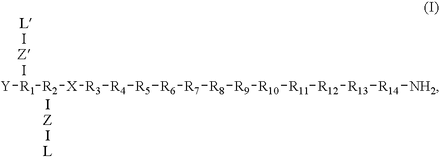

as well as pharmaceutically acceptable salts, derivatives and fragments thereof, wherein the substituents are as those disclosed in the specification. These compounds, and the pharmaceutical compositions containing them, are useful for the treatment of diseases such as, for example, obesity and diabetes.

### PRIORITY TO RELATED APPLICATION(S)

This application claims the benefit of U.S. Provisional Application No. 61/111,442, filed Nov. 5, 2008, which is hereby incorporated by reference in its entirety.

### RELATED APPLICATIONS

This application is related to U.S. application Ser. No. 12/547,076 filed Aug. 25, 2009, pending, which is expressly incorporated herein by reference.

This application is also related to U.S. application Ser. No. 11/328,743, filed Jan. 10, 2006, issued as U.S. Pat. No. 7,410,949; provisional application Ser. No. 60/444,840, filed Jan. 18, 2005; and U.S. application Ser. No. 12/136,263, filed Jun. 10, 2008, pending, all of which are expressly incorporated herein by reference.

This application is further related to U.S. application Ser. No. 11/607,230, filed Dec. 1, 2006, pending; and provisional application Ser. Nos. 60/855,249, filed Oct. 30, 2006, and 60/748,071, filed Dec. 7, 2005, all of which are incorporated herein by reference.

### FIELD OF THE INVENTION

The invention provides for truncated and lipidated analogs of PYY₃₋₃₆. The analogs are agonists of the neuropeptide-2 receptor and are useful for the treatment of metabolic diseases and disorders, such as, for example, obesity, type 2 diabetes, metabolic syndrome, insulin resistance and dyslipidemia.

All documents cited in this document are expressly incorporated herein by reference.

### BACKGROUND OF THE INVENTION

Metabolic diseases and disorders are widely recognized as serious health problems for developed countries, having reached epidemic levels in the United States. According to recent studies on obesity, for example, more than 50% of the U.S. population is considered overweight, with more than 25% diagnosed as clinically obese and at considerable risk for heart disease, type 2 diabetes and certain cancers. This epidemic presents a significant burden on the health care system as projected obesity treatment costs of more than $70 billion annually are expected in the U.S. alone. Strategies for treating obesity include reduction of food intake and enhancing the expenditure of energy.

Neuropeptide Y (NPY), a 36 amino acid peptide neurotransmitter, is a member of the pancreatic polypeptide class of neurotransmitters/neurohormones which has been shown to be present in both the periphery and central nervous system. NPY is one of the most potent orexogenic agents known and has been shown to play a major role in the regulation of food intake in animals, including humans.

Six NPY receptors, the Y1-, Y2-, Y3-, Y4, and Y5- and Y6-subtypes, have been cloned, which belong to the rhodopsin-like G-protein-coupled 7-transmembrane spanning receptors (GPCR). The NPY Y2 receptor (Y2R) is a 381 amino-acid receptor which inhibits the activation of adenyl cyclase via Gᵢ while displaying low homology with other known NPY receptors. There is a high degree of conservation between rat and human Y2 receptors with 98% amino acid identity.

The Y2R receptor is widely distributed within the central nervous system in both rodents and humans. In the hypothalamus, Y2 mRNA is localized in the arcuate nucleus, preoptic nucleus, and dorsomedial nucleus. In the human brain, Y2R is the predominant Y receptor subtype. Within the arcuate nucleus, over 80% of the NPY neurons co-express Y2R mRNA. Application of a Y2-selective agonist has been shown to reduce the release of NPY from hypothalamic slices in vitro, whereas the Y2 non-peptide antagonist BIIE0246 increases NPY release. These findings support the role of Y2R as a presynaptic autoreceptor that regulates the NPY release and hence may be involved in the regulation of feeding. (Kaga, T. et al., Peptides 22: 501-506 (2001) and King P J et al., Eur J Pharmacol 396: R1-3 (2000)).

Peptide YY₃₋₃₆ (PYY₃₋₃₆) is a 34 amino acid linear peptide having neuropeptide Y2 agonist activity. It has been demonstrated that Intra-arcuate (IC) or Intra-peritoneal (IP) injection of PYY₃₋₃₆ reduced feeding in rats and, as a chronic treatment, reduced body weight gain. Intra-venous (IV) infusion (0.8 pmol/kg/min) for 90 min of PYY₃₋₃₆ reduced food intake in obese and normal human subjects over 24 hours. These finding suggest that the PYY system may be a therapeutic target for the treatment of obesity. (Batterham R L et al., Nature 418: 650-654 (2002); Batterham R L et al., New Engl J Med 349: 941-948 (2003)). Further, a Cys²-(D)Cys²⁷-cyclized version of PYY, in which residues 5-24 were replaced by a methylene-chain of 5 to 8 carbons in length, showed activation of the intestinal PYY receptor, as evidenced by reduced current across voltage-clamped mucosal preparations of rat jejunum. (Krstenansky, et al. in Peptides, Proceedings of the Twelfth American Peptide Symposium. J. Smith and J. Rivier Editors, ESCOM. Leiden Page 136-137).

In addition, recent data have shown that Roux-enY gastric bypass patients have an early and exaggerated increase in PYY levels that may be partly responsible for the early glycemic control and long term weight maintenance demonstrating the importance of this peptide in the pathogenesis of metabolic diseases. Other known actions of PYY include: reduced gastric emptying and delayed gastrointestinal transit that is responsible for improved postprandial glycemic control. Indices of hyperglycaemia such as HbA_{1c} and fructosamine show a dose-dependent reduction after peripheral administration of PYY₃₋₃₆ in animal models of type 2 diabetes. Thus, these results indicate that PYY₃₋₃₆, or pharmaceutically related agonists, may offer a long term therapeutic approach to glycemic and weight control. (Korner et al., J Clin Endocrinol Metabol 90: 359-365 (2005); Chan J L et al., Obesity 14: 194-198 (2006); Stratis C et al., Obes Surg 16: 752-758 (2006); Borg C M et al., Br J Surg 93: 210-215 (2006); and Pittner R A et al., Int J Obes 28: 963-971 (2004)).

A need exists, therefore, for novel engineered analogs of PYY having lower molecular weight, while possessing equal or better potency and selectivity against Y1, Y4 and Y5 receptors, pharmacokinetic properties and pharmacological properties.

### SUMMARY OF THE INVENTION

Provided herein are neuropeptide-2 receptor agonists of formula (I):

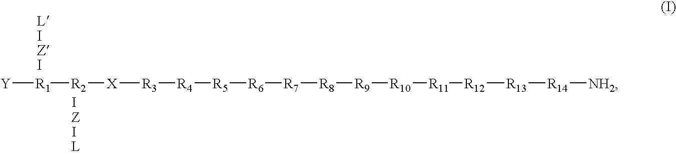

The compounds of the invention are preferably useful for treating metabolic diseases and disorders. Such metabolic diseases and disorders include, for example, obesity, diabetes, preferably type 2 diabetes, metabolic syndrome (also known as Syndrome X), insulin resistance, dyslipidemia, impaired fasting glucose and impaired glucose tolerance.

### DETAILED DESCRIPTION OF THE INVENTION

In one embodiment of the present invention, provided is a neuropeptide-2 receptor agonist of the formula (I):

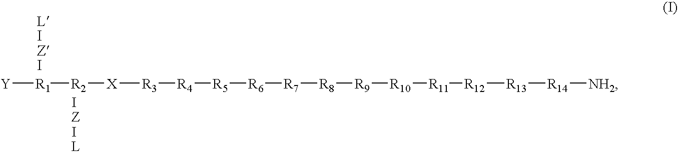

wherein:
L, L′ is a lipid moiety;
X is (4-oxo-6-piperazin-1-yl-4H-quinazolin-3-yl)-acetic acid (Pqa);
Y is H, an acyl moiety or gyro-Glu;
Z, Z′ is a spacer moiety or absent;

### R₁ is Ile, Ala, (D)Ile or N-methyl Ile;

R₂ is Lys, Ala, (D)Lys, N-methyl lys, Nle or (Lys-Gly);

### R₃ is Arg, Ala, (D)Arg, N-methyl Arg or Phe;

### R₄ is His, Ala, (D)His or N-methyl His;

### R₅ is Tyr, Ala, (D)Tyr, N-methyl Tyr or Trp;

### R₆ is Leu, Ala, (D)Leu or N-methyl Leu;

### R₇ is Asn, Ala or (D)Asn;

### R₈ is Leu or Trp;

### R₉ is Val, Ala, (D)Val or N-methyl Val;

### R₁₀ is Thr, Ala or N-methyl Thr;

### R₁₁ is Arg, (D)Arg or N-methyl Arg;

### R₁₂ is Gln or Ala;

### R₁₃ is Arg, (D)Arg or N-methyl Arg; and

### R₁₄ is Tyr, (D) Tyr, N-methyl Tyr, Phe or Trp,

wherein moieties L-Z- and L′-Z′- are not both present,
or a pharmaceutically acceptable salt thereof.

In a further embodiment of the present invention, provided is a pharmaceutical composition, comprising a therapeutically effective amount of the neuropeptide-2 receptor agonist according to formula I, or a salt thereof, and a pharmaceutically acceptable carrier.

The compounds of the invention are advantageous because, for example, they are truncated versions of the PYY₃₋₃₆. The shorter peptides, for example, not only facilitate easier synthesis and purification of the compounds, but also improve and reduce manufacturing procedures and expenses. Moreover, the compounds of the invention will preferably interact with Y2-receptors and not with homologous receptors such as NPY Y1, Y4 and Y5. Unwanted agonist or antagonist side reactions are, thereby, minimized. The truncated-lipidated peptides also exhibit longer half-life in vivo and favorable pharmacokinetic properties compared to native peptides while maintaining their biological activity and receptor specificity.

It is to be understood that the invention is not limited to the particular embodiments of the invention described herein, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Although any methods, devices and materials similar or equivalent to those described herein can be used in the practice or testing of the invention, the preferred methods, devices and materials are now described.

All peptide sequences mentioned herein are written according to the usual convention whereby the N-terminal amino acid is on the left and the C-terminal amino acid is on the right, unless noted otherwise. A short line between two amino acid residues indicates a peptide bond. Where the amino acid has isomeric forms, it is the L form of the amino acid that is represented unless otherwise expressly indicated. For convenience in describing this invention, the conventional and nonconventional abbreviations for the various amino acids are used. These abbreviations are familiar to those skilled in the art, but for clarity are listed below: Asp=D=Aspartic Acid; Ala=A=Alanine; Arg=R=Arginine; Asn=N=Asparagine; Gly=G=Glycine; Glu=E=Glutamic Acid; Gln=Q=Glutamine; His=H=Histidine; Ile=I=Isoleucine; Leu=L=Leucine; Lys=K=Lysine; Met=M=Methionine; Phe=F=Phenylalanine; Pro=P=Proline; Ser=S=Serine; Thr=T=Threonine; Trp=W=Tryptophan; Tyr=Y=Tyrosine; Cys=C=Cysteine; and Val=V=Valine.

Also for convenience, the following abbreviations or symbols are used to represent the moieties, reagents and the like used in this invention:

Pqa is (4-oxo-6-piperazin-1-yl-4H-quinazolin-3-yl)-acetic acid;
6-Ahx is 6-Aminohexanoic acid;

### Cha is Cyclohexylalanine;

### (1)NaI is 1-Naphtylalanine;

### (2)NaI is 2-Naphtylalanine;

### Alloc is Alloxycarbonyl;

### Fmoc is 9-Fluorenylmethyloxycarbonyl;

### Mtt is 4-Methyltrityl;

Pmc is 2,2,5,7,8-Pentamethylchroman-6-sulfonyl;
Pbf is 2,24,6,7-Pentamethyldihydro-benzofuran-5-sulfonyl
CH₂Cl₂ is Methylene chloride;
Ac₂O is Acetic anhydride;

### CH₃CN is Acetonitrile;

### DMAc is Dimethylacetamide;

### DMF is Dimethylformamide;

### DIPEA is N,N-Diisopropylethylamine;

TFA is Trifluoroacetic acid;
iPr₃SiH is Triisopropylsilane;

### HOBt is N-Hydroxybenzotriazole;

### DIC is N,N′-Diisopropylcarbodiimide;

BOP is Benzotriazol-1-yloxy-tris-(dimethylamino)phosphonium hexafluorophosphate;
HBTU is 2-(1H-Benzotriazole-1-yl)-1,1,3,3-tetramethyluronium hexafluorophosphate;
15-ATOPA is 15-amino-4,7,10,13,-tetraoxapentadecanoic acid
12-ATODA is 12-amino-4,7,10-trioxadodcadecanoic acid
8-ADOSA is N-(8-amino-3,6-dioxa-octyl)-succinamic acid
5-AOPSA is N-(5-amino-3-oxa-pentyl)-succinamic acid
NMP is 1-methyl 2-pyrrolidinone;
FAB-MS is Fast atom bombardment mass spectrometry; and
ES-MS is Electro spray mass spectrometry.

As used herein, the term “lipid moiety” means an optionally substituted linear or branched alkanoyl group of from 4-24 carbon atoms, preferably from 12-20 carbon atoms. The lipid moiety may be naturally-occurring or synthetic. Preferred lipid moieties include, but are not limited to, caproyl-, lauroyl-, myrisoyl-, palmitoyl-, 16-bromohexadecanoyl-, 2-hexyldecanoyl-, eicosanoyl-, and the like.

As used herein, the term “acyl” means an optionally substituted alkyl, cycloalkyl, heterocyclic, aryl or heteroaryl group bound via a carbonyl group and includes groups such as acetyl, propionyl, benzoyl, 3-pyridinylcarbonyl, 2-morpholinocarbonyl, 4-hydroxybutanoyl, 4-fluorobenzoyl, 2-naphthoyl, 2-phenylacetyl, 2-methoxyacetyl and the like.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a, saturated or unsaturated, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, bornyl, adamantyl, and the like. In a preferred embodiment, the “cycloalkyl” moieties can optionally be substituted with one, two, three or four substituents, with the understanding that said substituents are not, in turn, substituted further unless indicated otherwise. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexene optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate, with the understanding that said substituents are not, in turn, substituted further.

The term “lower alkyl”, alone or in combination with other groups, refers to a branched or straight-chain alkyl radical of one to nine carbon atoms, preferably one to six carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthalene, 1,2-dihydronaphthalene, indanyl, 1H-indenyl and the like.

The alkyl, lower alkyl and aryl groups may be substituted or unsubstituted. When substituted, there will generally be, for example, 1 to 4 substituents present, with the understanding that said substituents are not, in turn, substituted further unless indicated otherwise. These substituents may optionally form a ring with the alkyl, lower alkyl or aryl group they are connected with.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group.

The heteroaryl group described above may be substituted independently with one, two, or three substituents, with the understanding that said substituents are not, in turn, substituted further unless indicated otherwise.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms as well as all regioisomeric forms.

The present representative compounds may be readily synthesized by any known conventional procedure for the formation of a peptide linkage between amino acids. Such conventional procedures include, for example, any solution phase procedure permitting a condensation between the free alpha amino group of an amino acid or residue thereof having its carboxyl group and other reactive groups protected and the free primary carboxyl group of another amino acid or residue thereof having its amino group or other reactive groups protected.

Such conventional procedures for synthesizing the novel compounds of the present invention include for example any solid phase peptide synthesis method. In such a method the synthesis of the novel compounds can be carried out by sequentially incorporating the desired amino acid residues one at a time into the growing peptide chain according to the general principles of solid phase methods. Such methods are disclosed in, for example, Merrifield, R. B., J. Amer. Chem. Soc. 85, 2149-2154 (1963); Barany et al., The Peptides, Analysis, Synthesis and Biology, Vol. 2, Gross, E. and Meienhofer, J., Eds. Academic Press 1-284 (1980), which are incorporated herein by reference.

Common to chemical syntheses of peptides is the protection of reactive side chain groups of the various amino acid moieties with suitable protecting groups, which will prevent a chemical reaction from occurring at that site until the protecting group is ultimately removed. Usually also common is the protection of the alpha amino group on an amino acid or fragment while that entity reacts at the carboxyl group, followed by the selective removal of the alpha amino protecting group at allow a subsequent reaction to take place at that site. While specific protecting groups have been disclosed in regard to the solid phase synthesis method, it should be noted that each amino acid can be protected by a protective group conventionally used for the respective amino acid in solution phase synthesis.

Alpha amino groups may be protected by a suitable protecting group selected from aromatic urethane-type protecting groups, such as allyloxycarbonyl, benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropyloxycarbonyl, 9-fluorenylmethyloxycarbonyl (Fmoc) and p-methoxybenzyloxycarbonyl (Moz); aliphatic urethane-type protecting groups, such as t-butyloxycarbonyl (Boc), diisopropylmethyloxycarbonyl, and isopropyloxycarbonyl. Herein, Fmoc is most preferred for alpha amino protection.

Guanidino groups may be protected by a suitable protecting group such as nitro, p-toluenesulfonyl (Tos), (Z,) pentamethylchromanesulfonyl (Pmc), 4-Methoxy-2,3,6,-trimethylbenzenesulfonyl (Mtr), (Pmc), (Mtr) and (Pbf) are most preferred for arginine (Arg).

Epsilon amino groups may be protected by a suitable protecting group such as 2-chloro benzyloxycarbonyl (2-Cl—Z), 2-Bromo benztloxycarbonyl (2-Br—Z)- and t-butyloxycarbonyl (Boc). Boc is the most preferred for (Lys).

Hydroxyl groups (OH) may be protected by a suitable protecting group such as benzyl (Bzl), 2,6-dichlorobenzyl (2,6-diCl-Bzl), and tent.-Butyl (t-Bu), (t-Bu) is most preferred for (Tyr), (Ser) and (Thr).

The beta- and gamma-amide groups of Asn and Gln may be protected by a suitable protecting group such as 4-methyltrityl (Mtt), 2,4,6-trimethoxybenzyl (Tmob), 4,4-Dimethoxydityl Bis-(4-methoxyphenyl)-methyl (Dod) and Trityl (Trt). Trt is the most preferred for (Asn) and (Gln).

The indole group may be protected by a suitable protecting group selected from formyl (For), Mesityl-2-sulfonyl (Mts) and t-butyloxycarbonyl (Boc). Boc is the most preferred for (Trp).

The imidazole group may be protected by a suitable protecting group selected from Benzyl (Bzl), t-butyloxycarbonyl (Boc), and Trityl (Trt). Trt is the most preferred for (His).

The synthesis of the amino acid Pqa is described by J. Hutchinson et. al (J. Med. Chem. 1996, 39, 4583-4591). The Fmoc-Pqa derivative was purchased from NeoMPS, Inc. (San Diego Calif.)

All solvents, isopropanol (iPrOH), methylene chloride (CH₂Cl₂), dimethylformamide (DMF) and N-methylpyrrolinone (NMP) were purchased from Fisher or Burdick & Jackson and were used without additional treatment. Trifluoroacetic acid was purchased from Halocarbon or Fluka and used without further purification.

Diisopropylcarbodiimide (DIC) and diisopropylethylamine (DIPEA) was purchased from Fluka or Aldrich and used without further purification. Hydroxybenzotriazole (HOBT) dimethylsulfide (DMS) and 1,2-ethanedithiol (EDT) were purchased from Sigma Chemical Co. and used without further purification. Protected amino acids were generally of the L configuration and were obtained commercially from Bachem, or Neosystem. Purity of these reagents was confirmed by thin layer chromatography, NMR and melting point prior to use. Benzhydrylamine resin (BHA) was a copolymer of styrene −1% divinylbenzene (100-200 or 200-400 mesh) obtained from Bachem or Advanced Chemtech. Total nitrogen content of these resins were generally between 0.3-1.2 meq/g.

In a preferred embodiment, peptides were prepared using solid phase synthesis by the method generally described by Merrifield, (J. Amer. Chem. Soc., 85, 2149 (1963)), although other equivalent chemical synthesis known in the art could be used as previously mentioned. Solid phase synthesis is commenced from the C-terminal end of the peptide by coupling a protected alpha-amino acid to a suitable resin. Such a starting material can be prepared by attaching an alpha-amino-protected amino acid by an ester linkage to a p-benzyloxybenzyl alcohol (Wang) resin, or by an amide bond between an Fmoc-Linker, such as p-((R,S)-α-(1-(9H-fluoren-9-yl)-methoxyformamido)-2,4-dimethyloxybenzyl)-phenoxyacetic acid (Rink linker) to a benzhydrylamine (BHA) resin. Preparation of the hydroxymethyl resin is well known in the art. Fmoc-Linker-BHA resin supports are commercially available and generally used when the desired peptide being synthesized has an unsubstituted amide at the C-terminus.

Typically, the amino acids or mimetic are coupled onto the Fmoc-Linker-BHA resin using the Fmoc protected form of amino acid or mimetic, with 2-5 equivalents of amino acid and a suitable coupling reagent. After couplings, the resin may be washed and dried under vacuum. Loading of the amino acid onto the resin may be determined by amino acid analysis of an aliquot of Fmoc-amino acid resin or by determination of Fmoc groups by UV analysis. Any unreacted amino groups may be capped by reacting the resin with acetic anhydride and diispropylethylamine in methylene chloride.

The alpha amino Fmoc protecting groups are removed under basic conditions. Piperidine, piperazine or morpholine (20-40% v/v) in DMF may be used for this purpose. Preferably 40% piperidine in DMF is utilized.

Following the removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. For example, appropriate reagents for such syntheses are benzotriazol-1-yl-oxy-tri-(dimethylamino) phosphonium hexafluorophosphate (BOP), Bromo-tris-pyrrolidino-phosphonium hexafluorophosphate (PyBroP), 2-(1H-Benzotriazole-1-yl)-1,1,3,3-tetramethyluronium hexafluorophosphate (HBTU), and diisopropylcarbodiimide (DIC). Preferred here are HBTU and DIC. Other activating agents are described by Barany and Merrifield (in The Peptides, Vol. 2, J. Meienhofer, ed., Academic Press, 1979, pp 1-284) and may be utilized. Various reagents such as 1-hydroxybenzotriazole (HOBT), N-hydroxysuccinimide (HOSu) and 3,4-dihydro-3-hydroxy-4-oxo-1,2,3-benzotriazine (HODhBT) may be added to the coupling mixtures in order to optimize the synthetic cycles. Preferred here is HOBt.

For preparation of N-terminal acetyl derivatives, acetylation was carried out by treating the resin bound peptide with 20% acetic anhydride in DMF with 5% DIEA. For other N-terminal acylations, acylation was carried out using the corresponding carboxylic acid activated in-situ with DIC/HOBt for 30 minutes.

The protocol for a typical synthetic cycle is as follows:

Protocol 1

| Step | Reagent | Time | |
|---|---|---|---|
| 1 | DMF | 2 × 30 | sec. |
| 2 | 20% piperidine/DMF | 1 | min. |
| 3 | 20% piperidine/DMF | 15 | min. |
| 4 | DMF | 2 × 30 | sec. |
| 5 | iPrOH | 2 × 30 | sec. |
| 6 | DMF | 3 × 30 | sec. |
| 7 | Coupling | 60 min-18 hours. | |
| 8 | DMF | 2 × 30 | sec. |
| 9 | iPrOH | 1 × 30 | sec. |
| 10 | DMF | 1 × 30 | sec. |
| 11 | CH₂Cl₂ | 2 × 30 | sec. |

Solvents for all washings and couplings were measured to volumes of 10-20 mL/g resin. Coupling reactions throughout the synthesis were monitored by the Kaiser Ninhydrin test to determine extent of completion (Kaiser et at. Anal. Biochem. 34, 595-598 (1970)). Slow reaction kinetics was observed for Fmoc-Arg (Pmc) and for couplings to secondary amines by sterically hindered acids. Any incomplete coupling reactions were either recoupled with freshly prepared activated amino acid or capped by treating the peptide resin with acetic anhydride as described above. The fully assembled peptide-resins were dried in vacuum for several hours.

For most compounds, the blocking groups were removed and the peptide cleaved from the resin. For example, the peptide-resins were treated with 100 μL ethanedithiol, 100 μl dimethylsulfide, 300 μL, anisole, and 9.5 mL trifluoroacetic acid, per gram of resin, at room temperature for 180 min. Or alternately the peptide-resins were treated with 1.0 mL triisopropyl silane and 9.5 mL trifluoroacetic acid, per gram of resin, at room temperature for 180 min. The resin was filtered off and the filtrates were precipitated in chilled ethyl ether. The precipitates were centrifuged and the ether layer was decanted. The residue was washed with two or three volumes of Et₂O and recentrifuged. The crude products were dried under vacuum.

Purification of the crude peptides was preferably performed on Shimadzu LC-8A system by high performance liquid chromatography (HPLC) on a reverse phase C-18 Column (50×250 mm. 300 Å, 10-15 μm). The peptides were injected to the columns in a minimum volume of either 0.1 AcOH/H₂O or CH₃CH/H₂O. Gradient elution was generally started at 20% B buffer, 20%-80% B over 70 minutes, (buffer A: 0.1% TFA/H₂O, buffer B: 0.1% TFA/CH₃CN) at a flow rate of 50 mL/min. UV detection was made at 220/280 nm. The fractions containing the products were separated and their purity was judged on Shimadzu LC-10AT analytical system using reverse phase Ace C18 column (4.6×50 mol) at a flow rate of 2 mL/min., gradient (20-80%) over 10 min. (buffer A: 0.1% TFA/H₂O, buffer B: 0.1% TFA/CH₃CN)). Fractions judged to be of high purity were pooled and lyophilized.

Purity of the final products was checked by analytical HPLC on a reversed phase column as stated above. Purity of all products was judged to be approximately 95-99%. All final products were also subjected to fast atom bombardment mass spectrometry (FAB-MS) or electrospray mass spectrometry (ES-MS). All products yielded the expected parent M+H ions within acceptable limits.

The compounds of the present invention can be provided in the form of pharmaceutically acceptable salts. Examples of preferred salts are those formed with pharmaceutically acceptable organic acids, e.g., acetic, lactic, maleic, citric, malic, ascorbic, succinic, benzoic, salicylic, methanesulfonic, toluenesulfonic, trifluoroacetic or pamoic acid, as well as polymeric acids such as tannic acid or carboxymethyl cellulose, and salts with inorganic acids, such as hydrohalic acids (e.g., hydrochloric acid), sulfuric acid, or phosphoric acid and the like. Any procedure for obtaining a pharmaceutically acceptable salt known to a skilled artisan can be used.

In the practice of the method of the present invention, an effective amount of any one of the peptides of this invention or a combination of any of the peptides of this invention or a pharmaceutically acceptable salt thereof, is administered via any of the usual and acceptable methods known in the art, either singly or in combination. Administration can be, for example, once a day, once every three days or once a week. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Thus, the method of the present invention is practiced when relief of symptoms is specifically required or perhaps imminent. Alternatively, the method of the present invention is effectively practiced as continuous or prophylactic treatment.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as an “effective amount”. For example, the dose for intranasal administration is typically in the range of about 0.001 to about 0.1 mg/kg body weight. In humans, the preferred subcutaneous dose based on peptide content is from about 0.001 mg to about 100 mg; preferably from about 0.1 mg to about 15 mg.

The invention will now be further described in the Examples which follow, which are intended as an illustration only and do not limit the scope of the invention.

### EXAMPLES

### Example 1

### Preparation of Fmoc-Linker-BHA Resin

Benzhydrylamine copolystyrene-1% divinylbenzene cross-linked resin (10.0 g, 9.3 mequiv, 100-200 ASTM mesh, Advanced ChemTech) was swelled in 100 mL CH₂Cl₂, filtered and washed successively with 100 mL each of CH₂Cl₂, 6% DIPEA/CH₂Cl₂ (two times), CH₂Cl₂ (two times). The resin was treated with p-((R,S)-α-(1-(9H-fluoren-9-yl)-methoxyformamido)-2,4-dimethoxybenzyl)-phenoxyacetic acid (Fmoc-Linker) (7.01 g, 13.0 mmol), N-hydroxybenzotriazole (2.16 g, 16.0 mmol), and N,N′-diisopropylcarbodiimide (2.04 mL, 13.0 mmol) in 100 mL 25% DMF/CH₂Cl₂ for 24 hours at room temperature. The resin was filtered and washed successively with 100 mL each of CH₂Cl₂ (two times), isopropanol (two times), DMF, and CH₂Cl₂ (three times). A Kaiser Ninhydrin analysis was negative. The resin was dried under vacuum to yield 16.12 g of Fmoc-Linker-BHA resin. A portion of this resin (3.5 mg) was subjected to Fmoc deprotection and quantitative UV analysis which indicated a loading of 0.56 mmol/g.

### Example 2

### Protocol for the Synthesis of Peptides by Applied Biosystem 433A Synthesizer Using Fluorenylmethyloxycarbonyl (Fmoc) Chemistry

For a 0.25 mmol scale peptide synthesis by Applied Biosystem 433A synthesizer (Foster City, Calif.), the FastMoc 0.25 mmol cycles were used with either the resin sampling or non resin sampling, 41 mL reaction vessel. The Fmoc-amino acid resin was suspended with 2.1 g NMP, 2 g of 0.45M HOBT/HBTU in DMF and 2M DIEA, then transferred to the reaction vessel. The basic FastMoc coupling cycle was represented by “BADEIFD,” wherein each letter represents a module (as defined by Applied Biosystems). For example:

B represents the module for Fmoc deprotection using 20% Piperidine/NMP and related washes and readings for 30 min (either UV monitoring or conductivity); A represents the module for activation of amino acid in cartridges with 0.45 M HBTU/HOBt and 2.0 M DIEA and mixing with N₂ bubbling; D represents the module for NMP washing of resin in the reaction vessel; E represents the module for transfer of the activated amino acid to the reaction vessel for coupling; I represents the module for a 10 minute waiting period with vortexing on and off of the reaction vessel; and F represents the module for cleaning the cartridge, coupling for approximately 10 minutes and draining the reaction vessel. Couplings were typically extended by addition of module “I” once or multiple times. For example, double couplings were run by performing the procedure “BADEIIADEIFD.” Other modules were available such as c for methylene chloride washes and “C” for capping with acetic anhydride. Individual modules were also modifiable by, for example, changing the timing of various functions, such as transfer time, in order to alter the amount of solvent or reagents transferred. The cycles above were typically used for coupling one amino acid. For synthesizing tetra peptides, however, the cycles were repeated and strung together. For example, BADEIIADEIFD was used to couple the first amino acid, followed by BADEIIADEIFD to couple the second amino acid, followed by BADEIIADEIFD to couple the third amino acid, followed by BADEIIADEIFD to couple the fourth amino acid, followed by BIDDcc for final deprotection and washing.

### Example 3

### Preparation of H-Ile-Lys-Pro-Glu-Ala-Pro-Gly-Glu-Asp-Ala-Ser-Pro-Glu-Glu-Leu-Asn-Arg-Tyr-Tyr-Ala-Ser-Leu-Arg-His-Tyr-Leu-Asn-Leu-Val-The-Arg-Gln-Arg-Tyr-NH₂ (PYY₃₋₃₆)

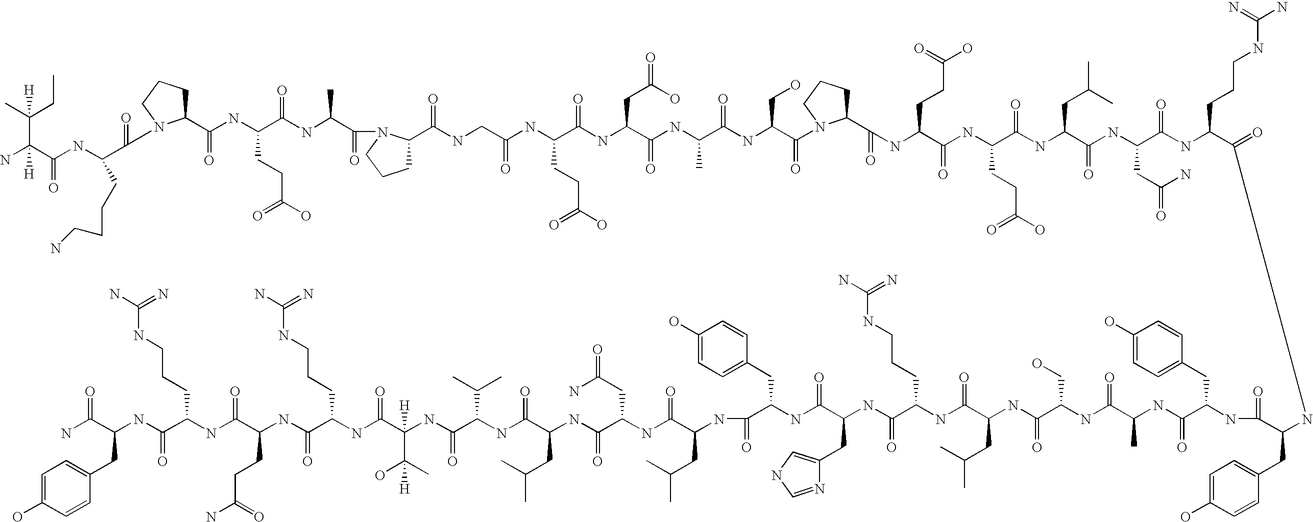

The above peptide was synthesized using Fmoc chemistry on an Applied Biosystem 433A synthesizer. The synthesizer was programmed for double coupling using the modules described in Example 2. The synthesis was carried out on a 0.25 mmol scale using the Fmoc-Linker-BHA resin (450 mg, 0.25 mmol) from Example 1. At the end of the synthesis, the resin was transferred to a reaction vessel on a shaker for cleavage. The peptide was cleaved from the resin using 13.5 mL 97% TFA/3% H2O and 1.5 mL triisopropylsilane for 180 minutes at room temperature. The deprotection solution was added to 100 mL cold ET₂O, and washed with 1 mL TFA and 30 mL cold Et₂O to precipitate the peptide. The peptide was centrifuged 2×50 mL polypropylene tubes. The precipitates from the individual tubes were combined in a single tube and washed 3 times with cold ET₂O and dried in a desiccator under house vacuum.

The crude material was purified by preparative HPLC on a Pursuit C18-Column (250×50 mm, 10 μm particle size) and eluted with a linear gradient of 2-70% B (buffer A: 0.1% TFA/H₂O; buffer B: 0.1% TFA/CH₃CN) in 90 min., flow rate 60 mL/min, and detection 220/280 nm. The fractions were collected and were checked by analytical HPLC. Fractions containing pure product were combined and lyophilized to yield 151 mg (15%) of a white amorphous powder. (ES)+-LCMS m/e calculated (calcd) for C₁₈₀H₂₇₉N₅₃O₅₄ 4049.55 found 4050.20.

### Example 4

### Preparation of Ac-Ile-Lys-Pqa-Arg-His-Tyr-Leu-Asn-Trp-Val-Thr-Arg-Gln-(NMe)Arg-Tyr-NH₂

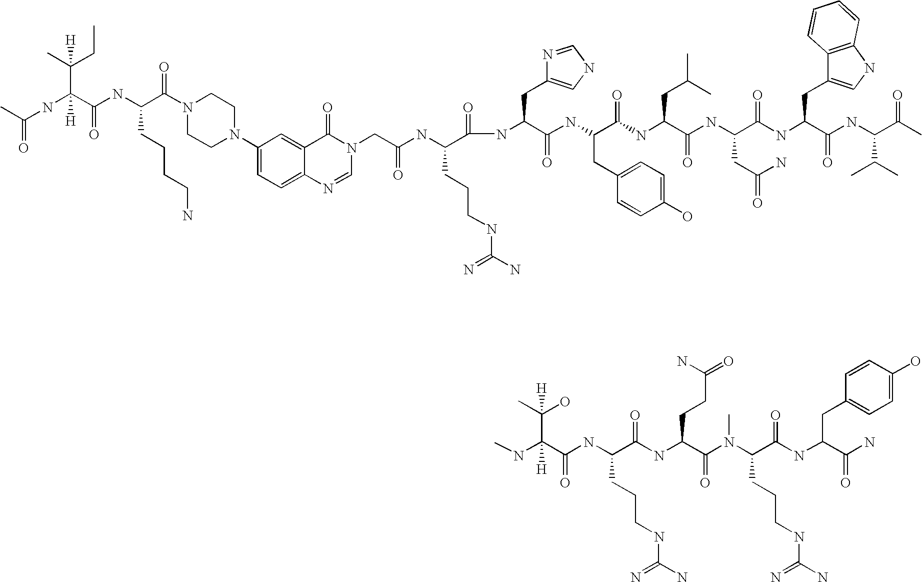

Fmoc-Linker-BHA resin (450 mg, 0.25 mmol) from Example 1 was subjected to solid phase synthesis and the crude peptide was purified following the procedure in Example 3 to yield 68 mg (12%) of white amorphous powder. (ES)+-LCMS m/e calculated (calcd) for C₁₀₆H₁₅₆N₃₄O₂₂ 2257.21 found 2257.19.

### Example 5

### Preparation of Ac-Ile-Lys(Butyryl)-Pqa-Arg-His-Tyr-Leu-Asn-Trp-Val-Thr-Arg-Gln-(NMe)Arg-Tyr-NH₂

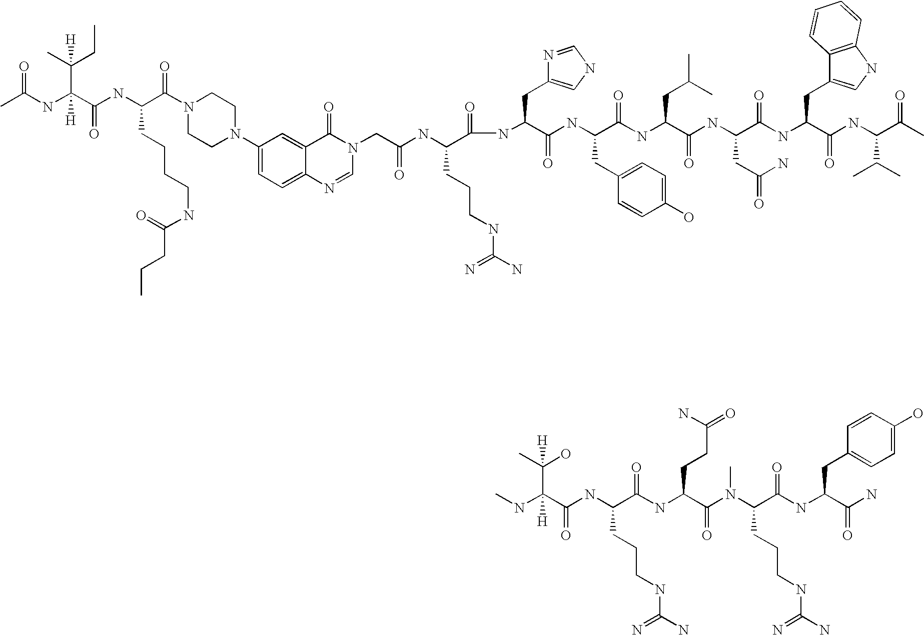

Ac-Ile-Lys-Pqa-Arg-His-Tyr-Leu-Asn-Trp-Val-Thr-Arg-Gln-Arg-Tyr-NH₂, 200 mg was dissolved in 5.0 mL DMF and 35 uL NMM and 250 uL Butyric anhydride was added. The solution was stirred for ˜16 hr (overnight). 3.0 mL 7 N NH₃ in MeOH was added and stirring continued for ½ hr. The product was then precipitated in 5.0 mL Et₂O, centrifuged, washed and dried in vacuo. The crude peptide was purified following the procedure in Example 3 to yield 18 mg (9%) of white amorphous powder. (ES)+-LCMS m/e calculated (calcd) C₁₁₀H₁₆₂N₃₄O₂₃ 2327.26 found 2327.26.

### Example 6

### Preparation of Ac-Ile-Lys(Capryloyl)-Pqa-Arg-His-Tyr-Leu-Asn-Trp-Val-Thr-Arg-Gln-(NMe)Arg-Tyr-NH₂

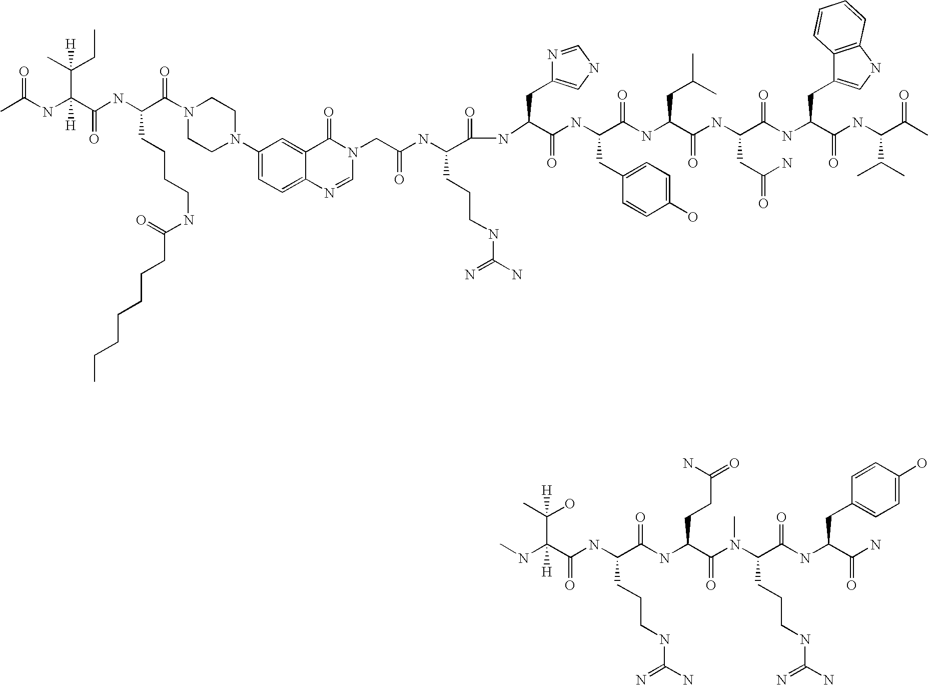

Ac-Ile-Lys-Pqa-Arg-His-Tyr-Leu-Asn-Trp-Val-Thr-Arg-Gln-Arg-Tyr-NH₂, 200 mg was dissolved in 5.0 mL DMF and N-hydroxybenzotriazole (425 mg, 3.15 mmol), DIEA (500 uL, 3.0 mmol) and capryloyl chloride (2.8 mL, 2.75 mmol) were reacted in 15 mL CH₂Cl₂ for 5 min and added to the peptide resin. The solution was stirred for 16 hr (overnight). 3.0 mL 7 N NH₃ in MeOH was added and stirring continued for ½ hr. The product was then precipitated in 5.0 mL Et₂O, centrifuged, washed and dried in vacuo. The crude peptide was purified following the procedure in Example 3 to yield 10 mg (5%) of a white amorphous powder. (ES)+-LCMS m/e calculated (calcd) C₁₁₄H₁₇₀N₃₄O₂₃ 2383.32 found 2383.32.

### Example 7

### Preparation of Ac-Ile-Lys(Lauroyl)-Pqa-Arg-His-Tyr-Leu-Asn-Trp-Val-Thr-Arg-Gln-(NMe)Arg-Tyr-NH₂

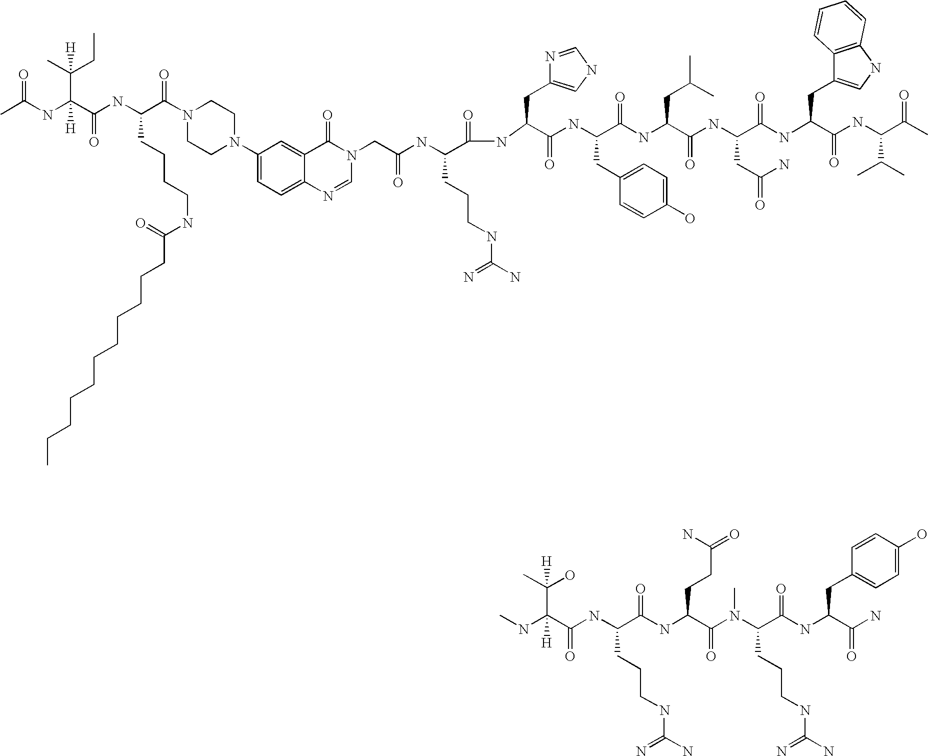

Ac-Ile-Lys-Pqa-Arg-His-Tyr-Leu-Asn-Trp-Val-Thr-Arg-Gln-Arg-Tyr-NH₂, 200 mg was dissolved in 5.0 mL DMF and N-hydroxybenzotriazole (425 mg, 3.150 mmol), DIEA (500 uL, 3.0 m) and lauroyl chloride (2.8 mL, 2.75 m) were reacted in 15 mL CH₂Cl₂ for 5 min and added to the peptide resin. The solution was stirred for 16 hr (overnight). 3.0 mL 7 N NH₃ in MeOH was added and stirring continued for ½ hr. The product was then precipitated in 5.0 mL Et₂O, centrifuged, washed and dried in vacuo. The crude material was purified by preparative HPLC on a Pursuit C18-Column (50×250 mm, 10 μm particle size) and eluted with a linear gradient of 20-90% B (buffer A: 0.1% TFA/H2O; buffer B: 0.1% TFA/CH3CN) in 90 min., flow rate 60 mL/min, and detection 220/280 nm. The fractions were collected and were checked by analytical HPLC. Fractions containing pure product were combined and lyophilized to yield 57 mg (26%) of white amorphous powder. (ES)+-LCMS m/e calculated (calcd) C₁₁₈H₁₇₈N₃₄O₂₃ 2439.38 found 2439.40.

### Example 8

### Preparation of Boc-Ile-Lys(TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin

Benzhydrylamine copolystyrene-1% divinylbenzene cross-linked resin (50.0 g, 55.0 mequiv, 100-200 ASTM mesh, Advanced ChemTech cat #SB5003) was swelled in 400 mL CH₂Cl₂, filtered and washed successively with 100 mL each of CH₂Cl₂, 6% DIPEA/CH₂Cl₂ (two times), CH₂Cl₂ (two times). The resin was treated with p-[(R,S)-α-[1-(9H-fluoren-9-yl)-methoxyformamido]-2,4-dimethoxybenzyl]-phenoxyacetic acid (Fmoc-Linker) (37.1 g, 69.0 mmol), N-hydroxybenzotriazole (9.356 g, 69.0 mmol), and N,N′-diisopropylcarbodiimide (55.0 mL, 300 mmol) in 400 mL DMF for 24 hours at room temperature.

The resin was filtered and washed successively with 400 mL each of CH₂Cl₂ (two times), isopropanol (two times), DMF, and CH₂Cl₂ (three times). A Kaiser Ninhydrin analysis was negative. Fmoc-Tyr(But)-OH (41.40 g., 90 mmol, N-hydroxbenzotriazole (12.2 g., 90.0 mmol) and N,N′-diisopropylcarbodiimide (55.0 mL, 300 mmol) in 400 mL DMF was added and allowed to react for 24 hours at room temperature. The reaction was not completed and, thus, 25.0 mL DIEA was added and the reaction was allowed to proceed for an additional 1½ hr. Coupling was still not complete, therefore acetylation with 25% Ac₂O, 5% DIEA in DMF for ¾ hr. was performed to obtain a negative ninhydrin (complete reaction). After washing and Fmoc removal, Fmoc-NMeArg(Mtr)-OH (43.0 g, 69.0 mmol), N-hydroxybenzotriazole (9.356 g, 69.0 mmol) and N,N′-diisopropylcarbodiimide (110.0 mL, 630 mmol) in 400 mL DMF was added, and allowed to react for 24 hours, whereby the reaction was completed. After washing and Fmoc removal, Fmoc-Gln(Trt)-OH (55.0 g., 90.0 mmol), N-hydroxbenzotriazole (12.2 g, 90.0 mmol) and N,N′-diisopropylcarbodiimide (55.0 mL, 300 mmol) in 400 mL DMF was added and the reaction was allowed to go for 24 h. The reaction was completed as determined by the chlorinal test.

The resin was washed and dried and 25.0 g (18.4%) was saved for different analogs. The remaining 110.0 g resin (44.6 mmol) was carried forward and 1.55 eqv. Fmoc-Arg(Pbf)-OH (45.0 g, 73.5 mmol), N-hydroxbenzotriazole (9.95 g, 73.5 mmol) and N,N′-diisopropylcarbodiimide (55.0 mL, 330 mmol) in 400 mL DMF was added, and the reaction was allowed to go for 24 hours at room temperature at which time, it was completed as judged by the ninhydrin test. After washing and Fmoc removal, Fmoc-Thr(But)-OH (27.40 g, 73.5 mmol), N-hydroxbenzotriazole. (9.95 g, 73.5 mmol) and N,N′-diisopropylcarbodiimide (55 mL, 300 mmole) in 400 mL DMF were added, and the reaction was allowed to go for 24 hours at room temperature at which time, it was completed as determined by the ninhydrin test. After washing and Fmoc removal Fmoc-Val-OH (23.6 g. 73.5 mmol), N-hydroxybenzotriazole (9.95 g, 73.5 mmol) and N,N′-diisopropylcarbodiimide (55.0 mL, 300 mmol) in 400 DMF was added and allowed to react for 6 hours at room temperature at which time, it was completed.

After washing and removal of the Fmoc, Fmoc-Trp-OH (29.5 0 g., 73.5 mmol), N-hydroxbenzotriazole (9.95 g, 73.5 mmol) and N,N′-diisopropylcarbodiimide (55.0 mL, 300 mmol) in 400 mL DMF was added. The reaction was complete after 6 hours. After washing and Fmoc removal, Fmoc-Asn(Trt)-OH (41.4 g, 73.5 mmol), N-hydroxbenzotriazole (9.95 g, 73.5 mmol) and N,N′-diisopropylcarbodiimide (55 mL, 300 mmol) in 400 mL DMF was added and allowed to react for 18 hours at room temperature at which time, it was completed.

After washing and Fmoc removal, Fmoc-Leu-OH (33.4 g, 73.5 mmol). N-hydroxbenzotriazole (9.95 g, 73.5 mmol) and N,N′-diisopropylcarbodiimide (55.0 mL, 300 mmol) in 400 mL DMF was added and allowed to react 6 hours. After washing and removal of the Fmoc, Fmoc-Tyr(But)-OH (41.4 0 g, 73.5 mmol), N-hydroxbenzotriazole (9.95 g, 73.5 mmol) and N,N′-diisopropylcarbodiimide (55.0 mL, 300 mmol) in 400 mL DMF was added. The reaction was complete after 18 hours. After washing and Fmoc removal, .Fmoc-His(Trt)-OH (55.5 g, 73.5 mmol), N-hydroxbenzotriazole (9.95 g, 73.5 mmol) and N,N′-diisopropylcarbodiimide (55.0 mL, 300 mmol) in 400 mL DMF was added. The reaction was complete after 20 hours. After washing and Fmoc removal, Fmoc-Arg(Pbf)-OH (58.4 g, 73.5 mmol), N-hydroxbenzotriazole (9.95 g, 73.5 mmol) and N,N′-diisopropylcarbodiimide (55.0 mL, 300 mmol) in 400 mL DMF was added. The reaction was complete after 20 hours.

After washing and removal of Fmoc, Fmoc-Pqa-OH (21.4 g, 73.5 mmol,) N-hydroxbenzotriazole (5.7 g, 42.05 mmol) and N,N′-diisopropylcarbodiimide (55.0 mL, 300 mmol) in 400 mL DMF was added. The reaction was complete after 16 hours. After washing and Fmoc removal, Fmoc-Lys(Alloc)-OH (18.5 g., 73.5 mmol) and N-hydroxbenzotriazole (9.95 g, 73.5 mmol) and N,N′-diisopropylcarbodiimide (55.0 mL, 300 mmol) in 400 mL DMF was added. The reaction was complete after 20 hours as determined by chlorinal test. After washing and drying, a portion was saved for coupling with Fmoc-Ile for N-acetylated analogs. The remaining peptide resin was treated with Boc-Ile-OH (25.0 g, 73.5 mmol) N-hydroxbenzotriazole (9.95 g, 73.5 mmol) and N,N′-diisopropylcarbodiimide (55.0 mL, 300 mmol) in 400 mL DMF for 20 hours at room temperature. The reaction was complete

Removal of the Aloc group from the epsilon-amino group of Lys: Argon was bubbled through a mixture of 1.2 g PdCl₂ (triphenylphosphine)₂, 5.0 mL morpholine, and 10.0 mL of acetic acid, then 25.0 mL Bu₃SnH was added. Bubbling with Ar was continued until the yellow solution become reddish brown. The reaction mixture was then shaken for ½ hr, and washed 3 times with DMF. The above procedure was repeated a second time (this time the mixture turned dark brown to almost black in color) and shaking was continued for ½ to ¾ hr. The resin was washed 2 times with DMF, 2 times with 5% DIEA/DMF and 3 times with DMF/CH₂Cl₂. The free epsilon-amine of Lysine was converted to the TFA salt by washing with 2.35 mL TFA added to CH₂Cl₂. The resin was then washed 2 times with CH₂Cl₂ and 4 times with MeOH and dried to constant weight under vacuo.

### Example 9

### Preparation of H-Ile-Lys(Lauroyl-6-Ahx)-Pqa-Arg-His-Tyr-Leu-Asn-Trp-Val-Thr-Arg-Gln-(NMe)Arg-Tyr-NH₂

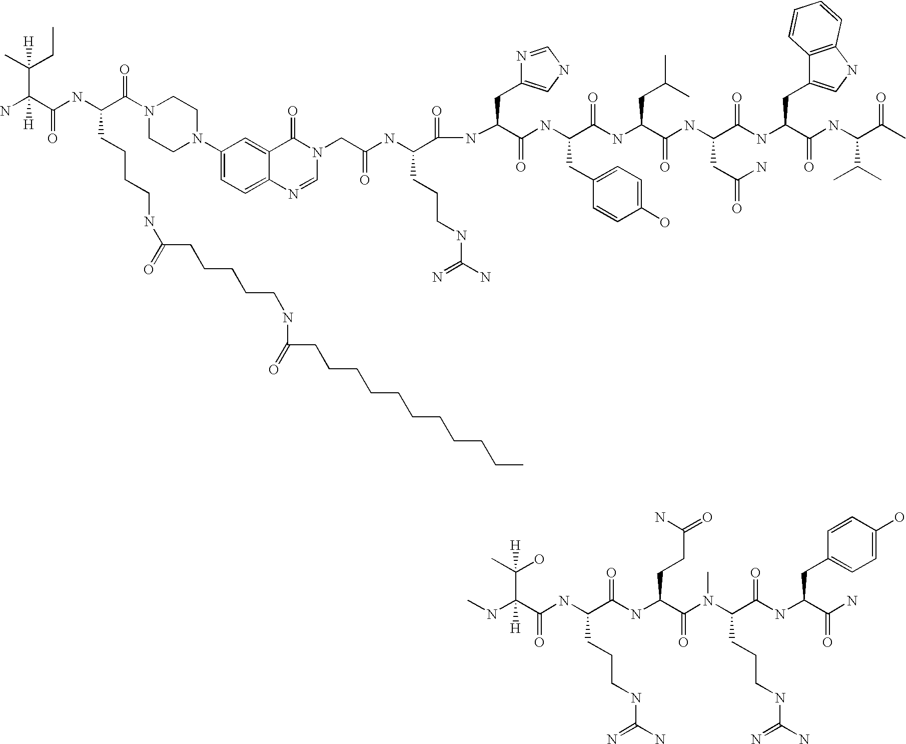

Boc-Ile-Lys(TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF N-hydroxybenzotriazole (425 mg, 3.150 mmol), DIEA (500 uL, 3.0 m) and Lauroyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 30 mg (7%) of white amorphous powder. (ES)+-LCMS m/e calculated (calcd) C₁₂₂H₁₈₇N₃₅O₂₃ 2510.45 found 2510.44.

### Example 10

### Preparation of H-Ile-Lys(Lauroyl-beta-Ala)-Pqa-Arg-His-Tyr-Leu-Asn-Trp-Val-Thr-Arg-Gln-(NMe)Arg-Tyr-NH₂

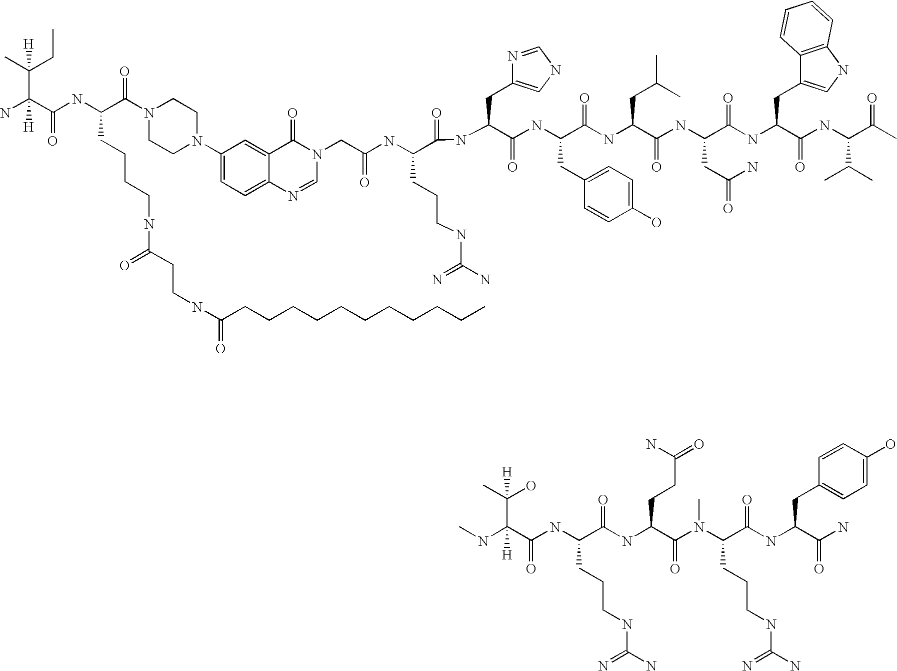

Boc-Ile-Lys (epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-betaAla (325.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and lauroyl chloride (2.8 mL, 2.75 mmol) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 20 mg (4%) of white amorphous powder. (ES)+-LCMS m/e calculated (calcd) C₁₁₉H₁₈₁N₃₅O₂₃ 2468.41, found 2468.6.

### Example 11

### Preparation of H-Ile-Lys(Lauroyl-Glu)-Pqa-Arg-His-Tyr-Leu-Asn-Trp-Val-Thr-Arg-Gln-(NMe)Arg-Tyr-NH₂

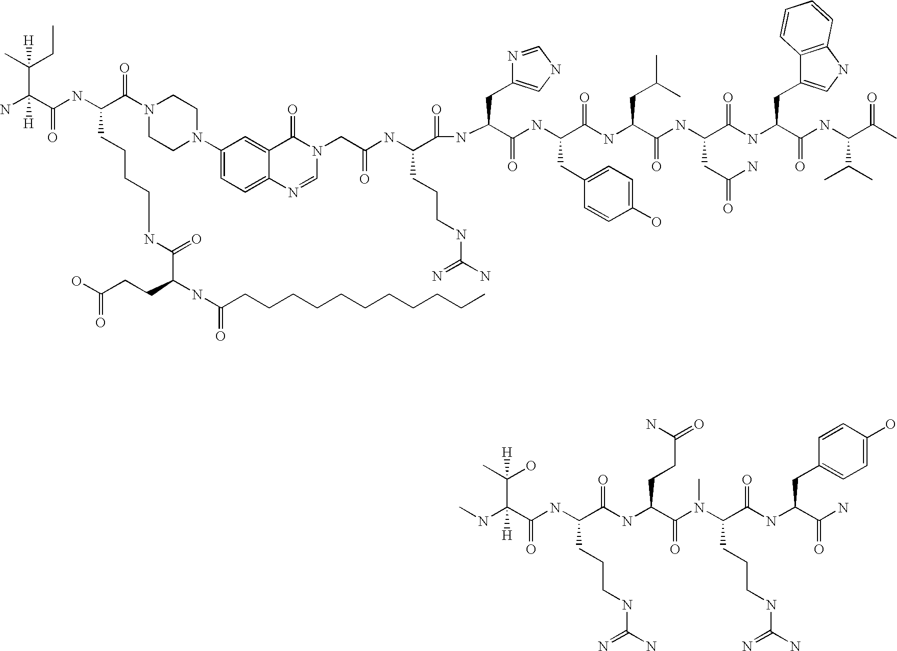

Boc-Ile-Lys(epsilon-TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-Glu(Bu^{t}) (325.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, N-hydroxybenzotriazole (425 mg, 3.150 mmol), DIEA (500 uL, 3.0 mmol) and lauroyl chloride (2.8 mL, 2.75 mmol) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 30 mg (7%) of white amorphous powder. (ES)+-LCMS m/e calculated (calcd) C₁₂₁H₁₈₃N₃₅O₂₅ 2526.41, found 2526.40.

### Example 12

### Preparation of H-Ile-Lys(Myristoyl-6Ahx)-Pro-Pqa-Arg-His-Tyr-Leu-Asn-Trp-Val-Thr-Arg-Gln-(NMe)Arg-Tyr-NH₂

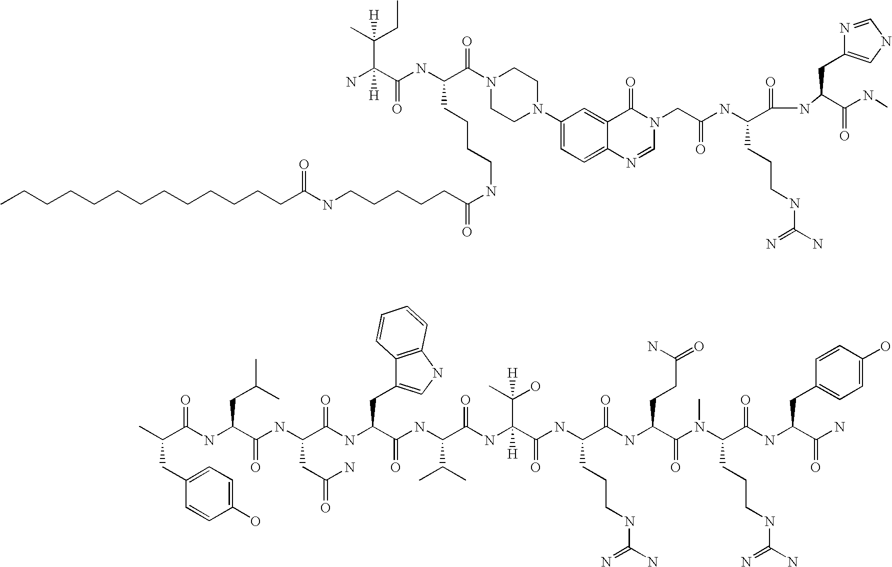

Boc-Ile-Lys(epsilonTFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-6-aminohexanoic acid (355 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, myristric acid (230 mg, 1 mmol); N-hydroxybenzotriazole (150 mg, 1.11 mmol), and N,N′-diisopropylcarbodiimide (1.50 mL, 2.0 mmol) were coupled and stirred over night. After washing with DMF 2 times and CH₂Cl₂ 3 times, cleavage was effected with TFA, 17 mL 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 66 mg (13%) of white amorphous powder. (ES)+-LCMS m/e calculated (calcd) C₁₂₄H₁₉₁N₃₅O₂₃ 2538.49, found 2538.47.

### Example 13

### Preparation of Ac-Ile-Lys(Palmitoyl)-Pqa-Arg-His-Tyr-Leu-Asn-Trp-Val-Thr-Arg-Gln-(NMe)Arg-Tyr-NH₂

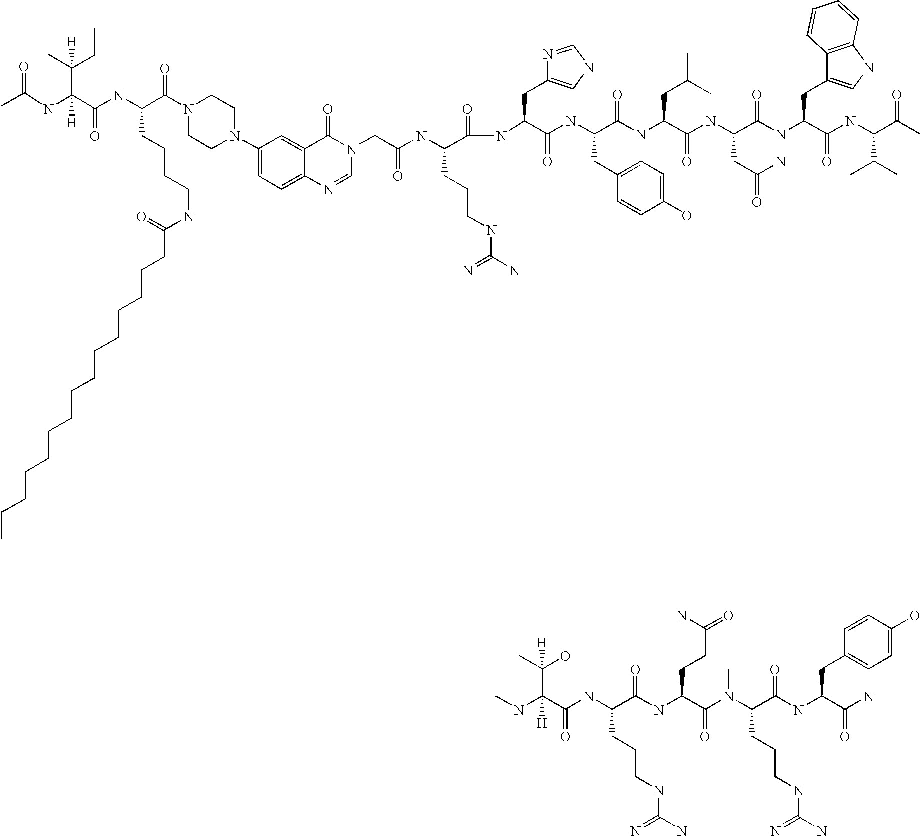

Ac-Ile-Lys-Pqa-Arg-His-Tyr-Leu-Asn-Trp-Val-Thr-Arg-Gln-Arg-Tyr-NH₂, 200 mg was dissolved in 5.0 mL DMF and N-hydroxybenzotriazole (425 mg, 3.15 mmol), DIEA (500 uL, 3.0 mmol) and palmitoyl chloride (2.8 mL, 2.8 mmol) were reacted in 15 mL of CH₂Cl₂ for 5 min. and added to the peptide resin. The solution was stirred for ˜16 hr (overnight). 3.0 mL 7 N NH₃ in MeOH was added and stirring continued for ½ hr. The product was then precipitated in 5.0 mL Et₂O, centrifuged, washed and dried in vacuo. The crude peptide was purified following the procedure in Example 7 to yield 42 mg (19%) of white amorphous powder. (ES)+-LCMS m/e calculated (calcd) C₁₂₂H₁₈₆N₃₄O₂₃ 2495.44, found 2495.43.

### Example 14

### Preparation of H-Ile-Lys(Palmitoyl)-Pqa-Arg-His-Tyr-Leu-Asn-Trp-Val-Thr-Arg-Gln-(NMe)Arg-Tyr-NH₂

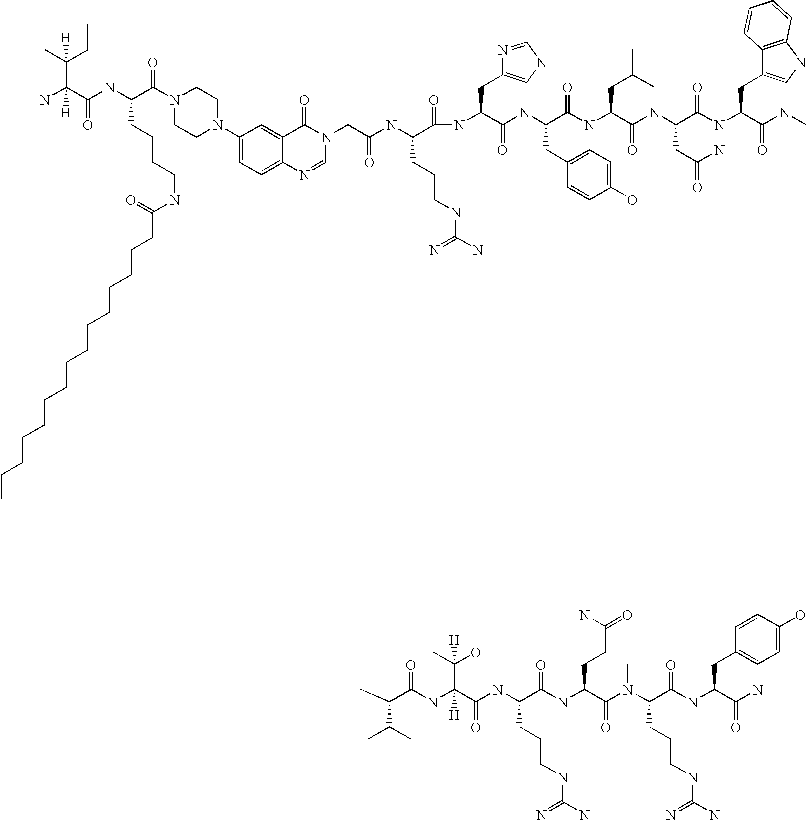

Boc-Ile-Lys(TFA epsilon salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 mmol) and palmitoyl chloride (2.8 mL, 2.8 mmol) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 46 mg (10%) of white amorphous powder. (ES)+-LCMS m/e calculated (calcd) C₁₂₀H₁₈₄N₃₄O₂₂ 2453.43, found 2453.41.

### Example 15

### Preparation of Palmitoyl-Ile-Lys-Pqa-Arg-His-Tyr-Leu-Asn-Trp-Val-Thr-Arg-Gln-(NMe)Arg-Tyr-NH₂

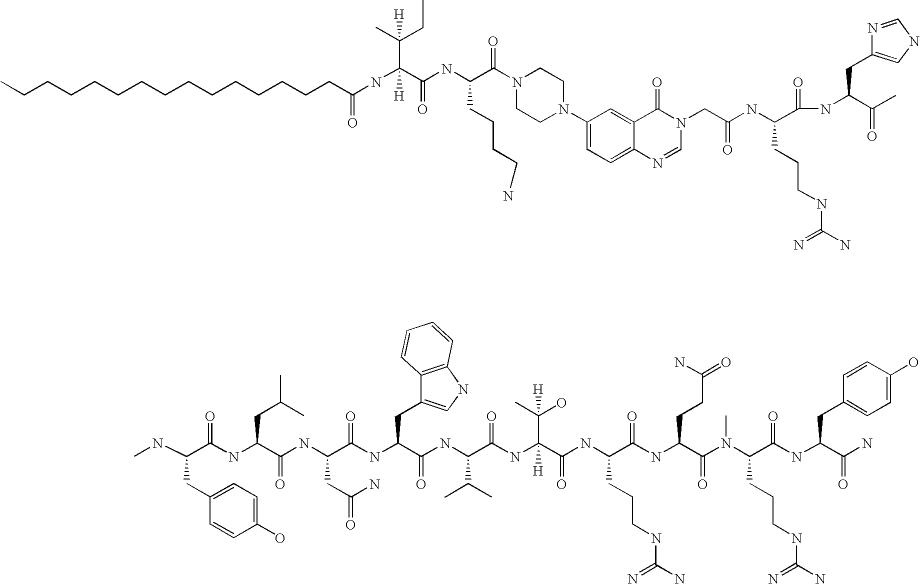

Fmoc-Linker-BHA resin (450 mg, 0.25 mmol) from Example 1 was subjected to solid phase synthesis. Synthesis was carried out according to the general procedure described in example 4 as far as the N-terminal deprotected 15-mer and acylated manually with palmitoyl chloride (288 uL, 1.0 mmol) and DIEA (200 uL, 1.15 mmol) in CH₂Cl₂ for ½ hr. The resin was cleaved and the product purified by following the procedure in Example 7 to yield 55 mg (9%) of white amorphous powder. (ES)+-LCMS m/e calculated (calcd) for C₁₂₀H₁₈₄N₃₄O₂₂ 2453.43, found 2453.41.

### Example 16

### Preparation of Palmitoyl-6-Ahx-Ile-Lys-Pqa-Arg-His-Tyr-Leu-Asn-Trp-Val-Thr-Arg-Gln-(NMe)Arg-Tyr-NH₂

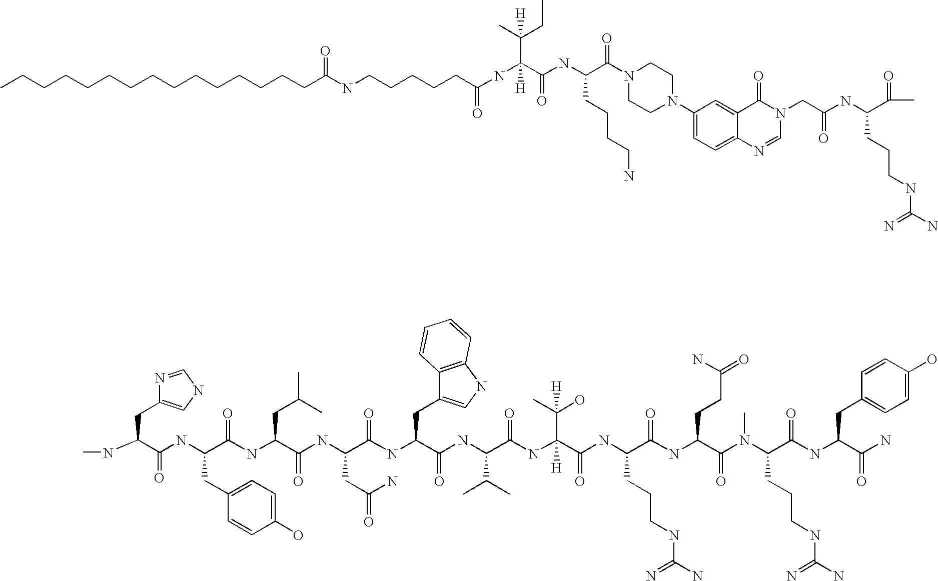

Fmoc-Linker-BHA resin (450 mg, 0.25 mmol) from Example 1 was subjected to solid phase synthesis. Synthesis was carried out as generally described in Example 4 as far as the deprotected 15 mer and coupled manually with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) were added and the reaction was allowed to proceed overnight. After Fmoc removal, the resin bound peptide was acylated with palmitoyl chloride (288 uL 1.0 mmol), DIEA (200 uL, 1.15 mmol) in CH₂Cl₂ for ½ hr. The resin was cleaved and the crude peptide was purified following the procedure in Example 7 to yield 45 mg (7%) of white amorphous powder. (ES)+-LCMS m/e calculated (calcd) for C₁₂₆H₁₉₅N₃₅O₂₃ 2566.52, found 2566.51.

### Example 17

### Preparation of Palmitoyl-6-Ahx-Ile-Lys-Pqa-Arg-His-Tyr-Leu-Asn-Trp-Val-Thr-Arg-Gln-Arg-Tyr-NH₂

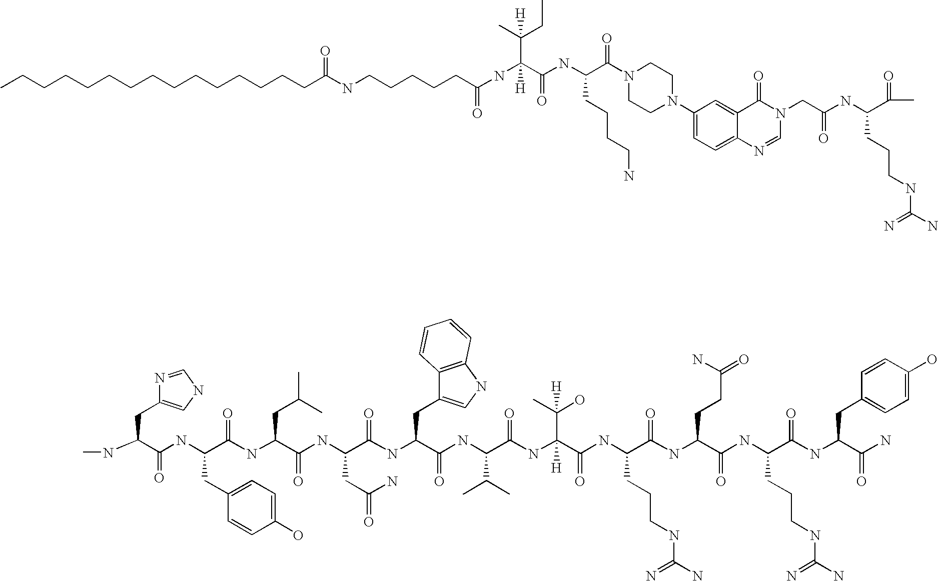

Fmoc-Linker-BHA resin (450 mg, 0.25 mmol) from Example 1 was subjected to solid phase synthesis. Synthesis was carried out according to the general procedure described in Example 4 as far as the deprotected 15-mer and coupled manually with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal the resin bound peptide was acylated with palmitoyl chloride (288 uL, 1.0 mmol) and DIEA (200 uL, 1.15 mol) in CH₂Cl₂ for ½ hr. The resin was cleaved and the crude peptide was purified following the procedure in Example 7 to yield 77 mg (12%) of white amorphous powder. (ES)+-LCMS m/e calculated (calcd) for C₁₂₅H₁₉₃N₃₅O₂₃ 2552.50 found 2552.49.

### Example 18

### Preparation of H-Ile-Lys(Palmitoyl-6-Ahx)-Pqa-Arg-His-Tyr-Leu-Asn-Trp-Val-Thr-Arg-Gln-(NMe)Arg-Tyr-NH₂

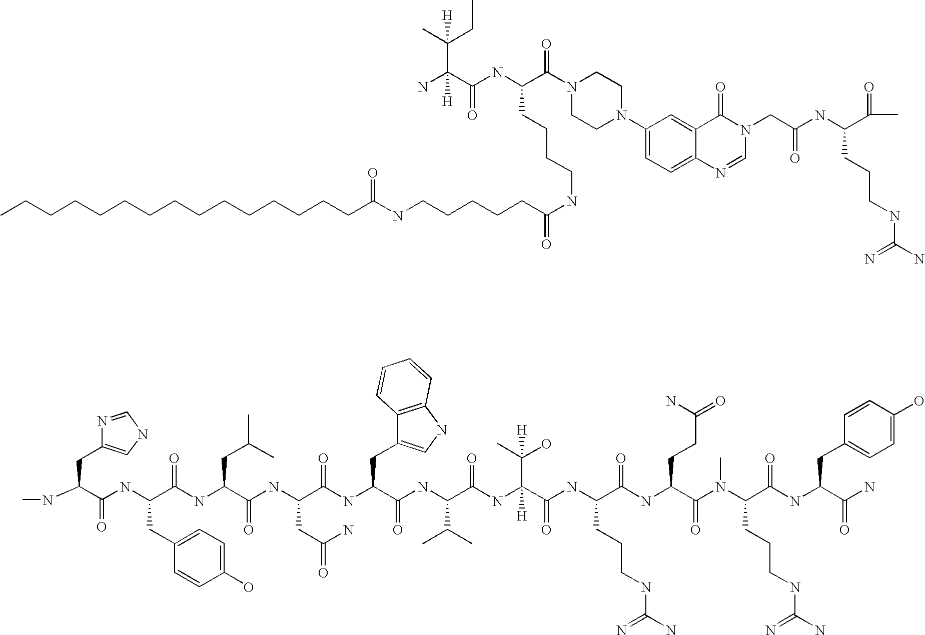

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 14 mg (3%) of white amorphous powder. (ES)+-LCMS m/e calculated (calcd) C₁₂₆H₁₉₅N₃₅O₂₃ 2566.51 found 2566.50.

### Example 19

### Preparation of H-Ile-Lys(Palmitoyl-6Ahx)-Pqa-Arg-His-Tyr-Leu-Asn-Trp-Val-Thr-Arg-Gln-Arg-Tyr-NH₂

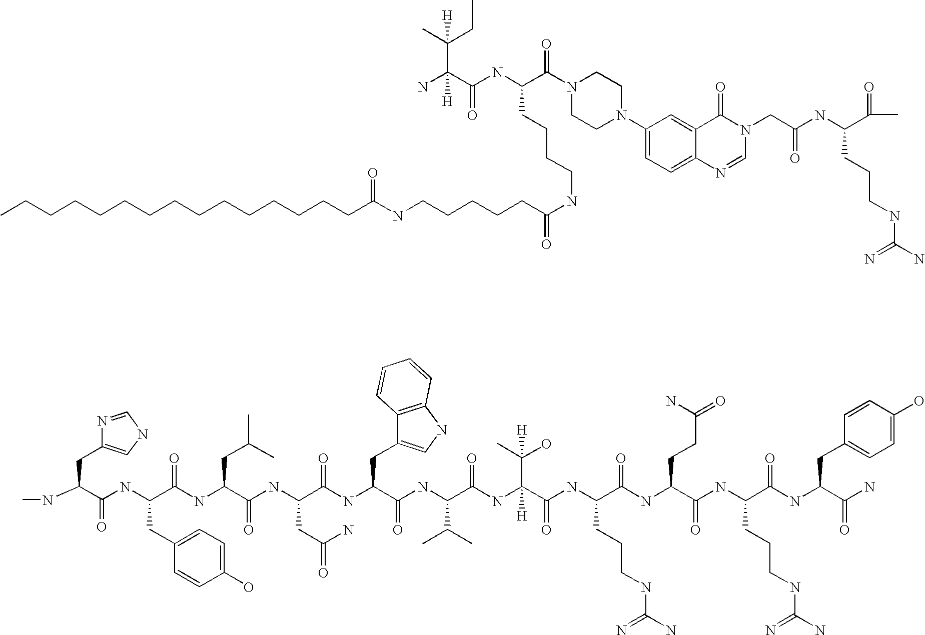

Boc-Ile-Lys(alloc)-Pqa-Arg(Pbf)-His(Trt)-Tyr(tBu)-Leu-Asn(Trt)-Trp-Val-Thr(tBu)-Arg(Pbf)-Gln(Trt)-Arg-Tyr(tBu)-Knorr resin (prepared as in Example 14) was washed with 5% DIEA in DMF and coupled with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and Palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 52 mg (15%) of white amorphous powder. (ES)+-LCMS m/e calculated (calcd) C₁₂₅H₁₉₃N₃₅O₂₃ 2552.50 found 2552.49.

### Example 20

### Preparation of H-Ile-Lys(Palmitoyl-beta-Ala)-Pqa-Arg-His-Tyr-Leu-Asn-Trp-Val-Thr-Arg-Gln-(NMe)Arg-Tyr-NH₂

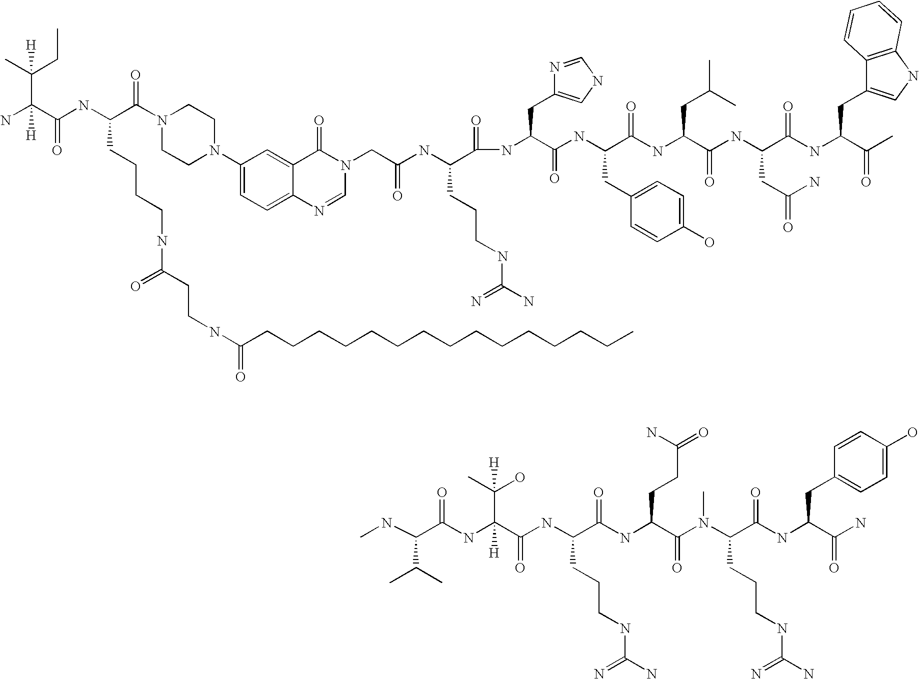

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-beta-Ala (312.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 20 mg (4.4%) of white amorphous powder. (ES)+-LCMS m/e calculated (calcd) C₁₂₃H₁₈₉N₃₅O₂₃ 2524.476, found 2524.47.

### Example 21

### Preparation of H-Ile-Lys(Palmitoyl-Glu)-Pqa-Arg-His-Tyr-Leu-Asn-Trp-Val-Thr-Arg-Gln-(NMe)Arg-Tyr-NH₂

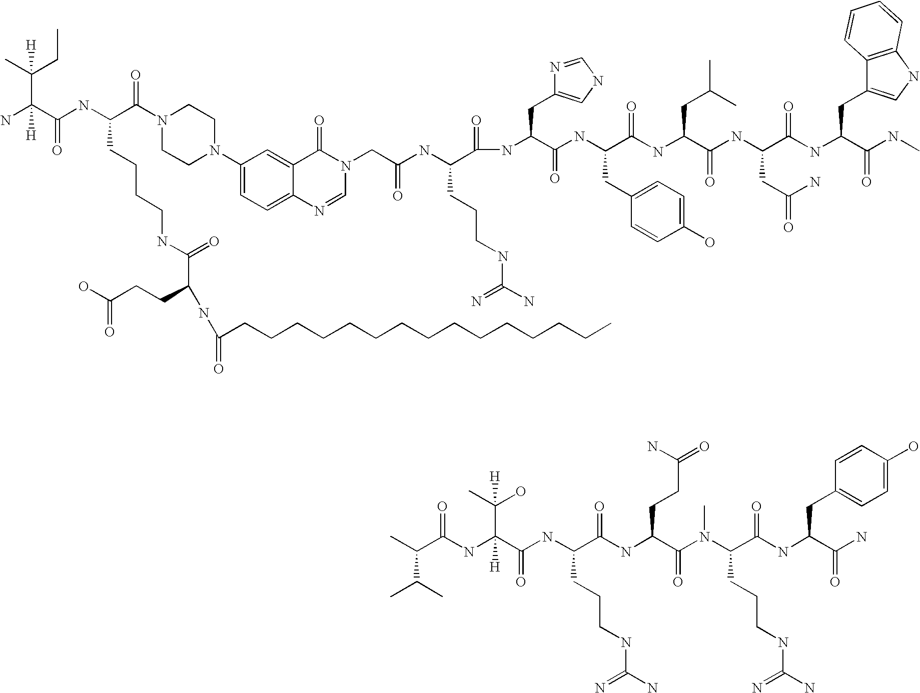

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-Glu(Bu^{t}) (312.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 14 mg (3%) of white amorphous powder. (ES)+-LCMS m/e calculated (calcd) C₁₂₅H₁₉₁N₃₅O₂₅ 2582.48, found 2582.48.

### Example 22

### Preparation of H-Ile-Lys(Palmitoyl-beta-Ala-Glu)-Pqa-Arg-His-Tyr-Leu-Asn-Trp-Val-Thr-Arg-Gln-(NMe)Arg-Tyr-NH₂

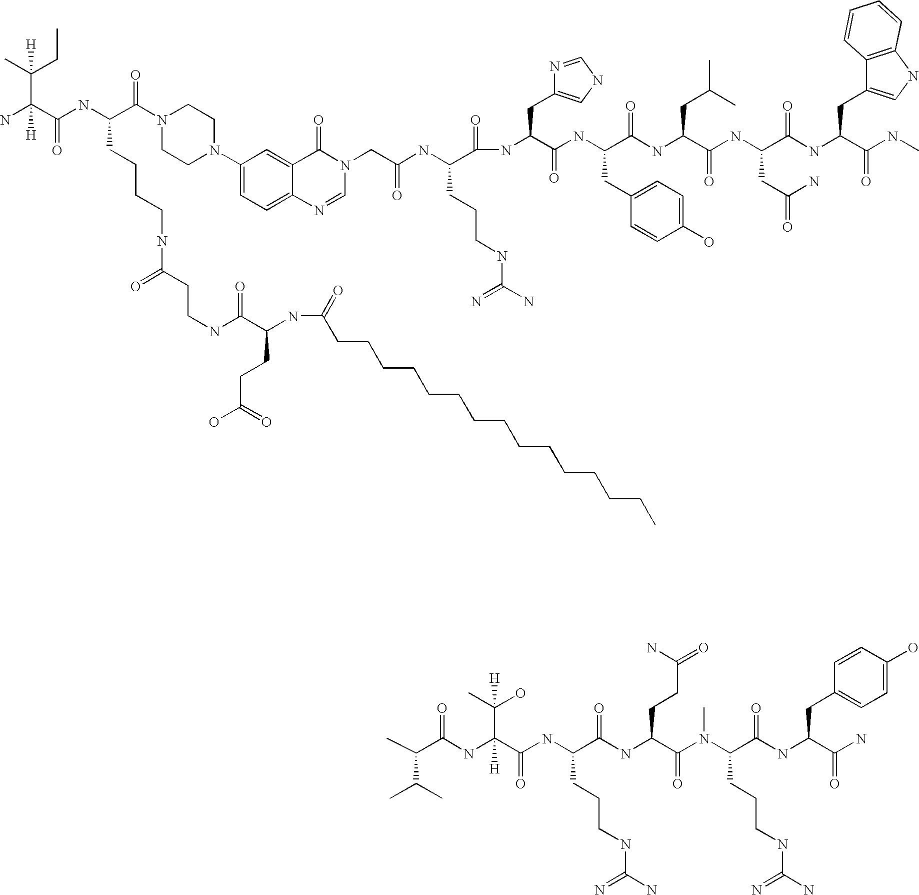

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-beta-Ala (312.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, Fmoc-Glu(Bu^{t}) (312.0 mg; 1.0 mmol), N-hydroxybenzotriazole (150 mg, 1.11 mmol), and N,N′-diisopropylcarbodiimide (1.50 mL, 2.0 mmol) were added and the reaction was allowed to procede overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 29 mg (6%) of white amorphous powder. (ES)+-LCMS m/e calculated (calcd) C₁₂₈H₁₉₆N₃₆O₂₆ 2653.51, found 2653.50.

### Example 23

### Preparation of H-Ile-Lys(Palmitoyl-Glu-Glu-)-Pqa-Arg-His-Tyr-Leu-Asn-Trp-Val-Thr-Arg-Gln-(NMe)Arg-Tyr-NH₂

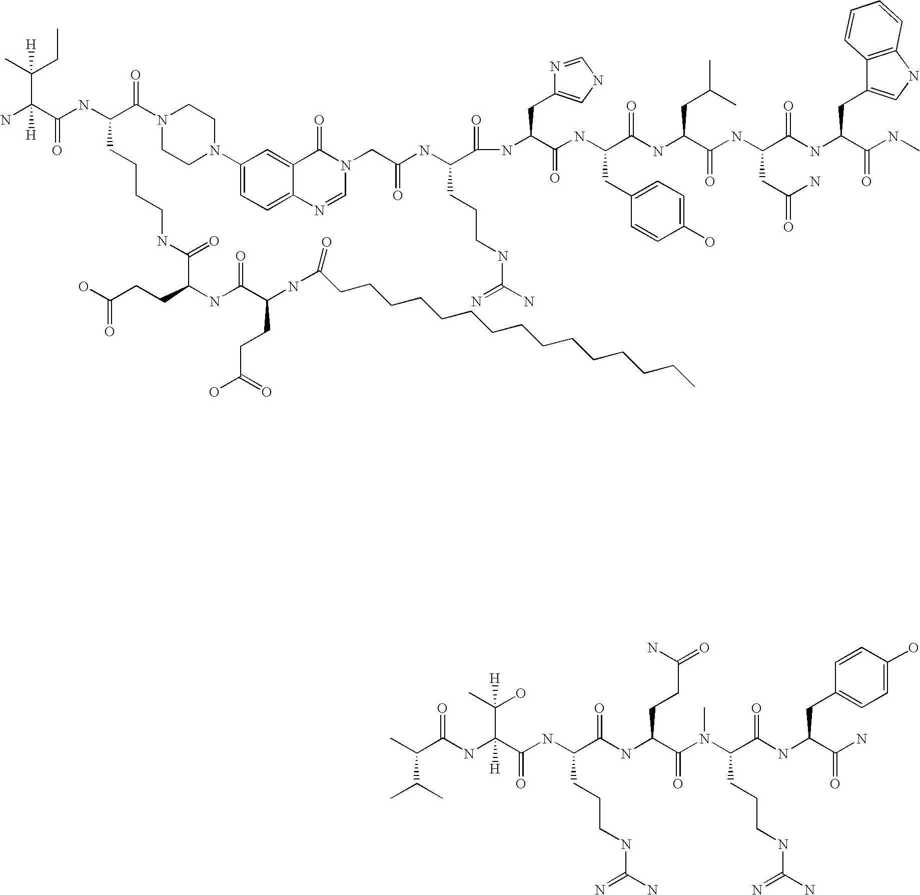

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-Glu(Bu^{t}) 426.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF coupling was effected with Fmoc-Glu(Bu^{t}) (426.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 25 mg (5%) of white amorphous powder. (ES)+-LCMS m/e calculated (calcd) C₁₃₀H₁₉₈N₃₆O₂₈ 2711.52, found 2711.51.

### Example 24

### Preparation of H-Ile-Lys(Palmitoyl-gamma-Glu)-Pqa-Arg-His-Tyr-Leu-Asn-Trp-Val-Thr-Arg-Gln-(NMe)Arg-Tyr-NH₂

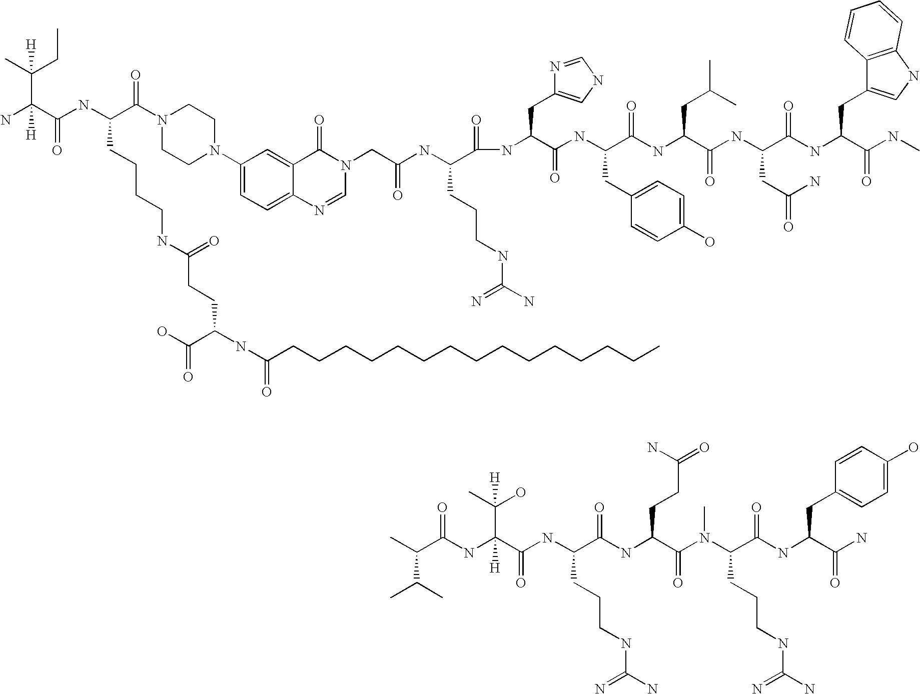

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-gamma-Glu-alpha OBu^{t} (426.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 28 mg (6%) of white amorphous powder. (ES)+-LCMS m/e calculated (calcd) C₁₂₅H₁₉₁N₃₅O₂₅ 2582.48, found 2582.47.

### Example 25

### Preparation of H-Ile-Lys(Palmitoyl-gamma-Glu-gamma-Glu-)-Pqa-Arg-His-Tyr-Leu-Asn-Trp-Val-Thr-Arg-Gln-(NMe)Arg-Tyr-NH₂

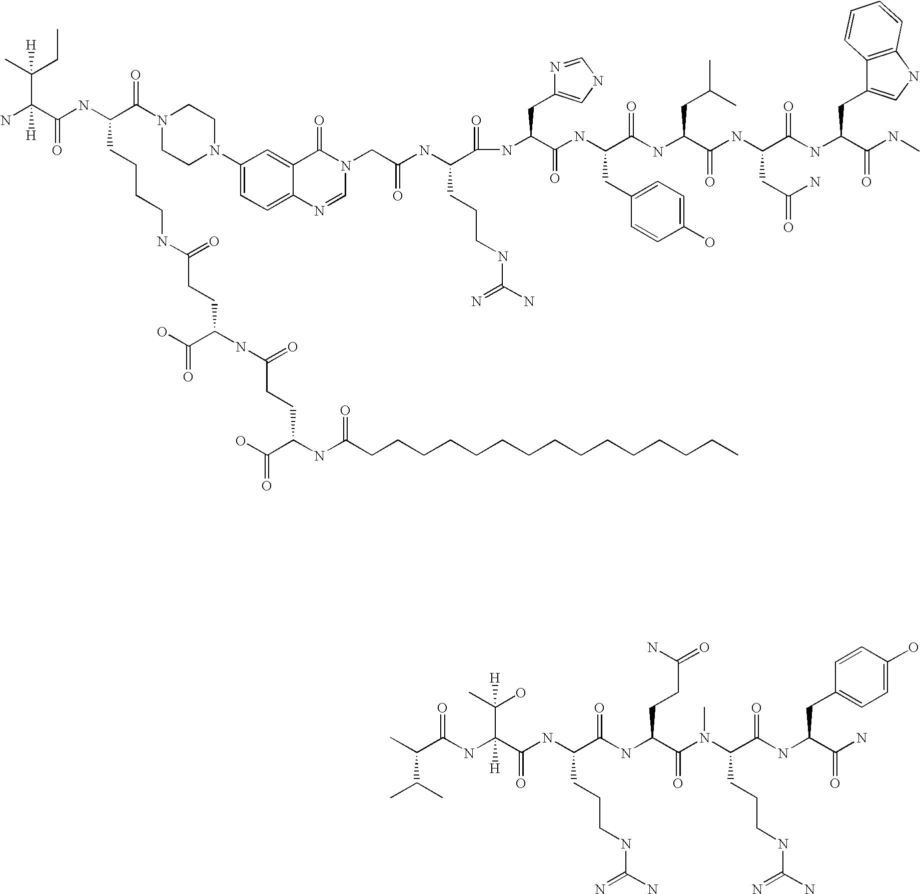

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-Glu-alpha-OBu^{t} (426.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, Fmoc-Glu-alpha-OBu^{t} (426.0 mg; 1.0 mmol), N-hydroxybenzotriazole (150 mg, 1.11 mmol), and N,N′-diisopropylcarbodiimide (1.50 mL, 2.0 mmol) were added and the reaction was allowed to proceed overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ times before cleavage with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 40 mg (8%) of white amorphous powder. (ES)+-LCMS m/e calculated (calcd) C₁₃₀H₁₉₈N₃₆O₂₈ 2711.52, found 2711.50.

### Example 26

### Preparation of H-Ile-Lys(Palmitoyl-beta-Ala-gamma-Glu-)-Pqa-Arg-His-Tyr-Leu-Asn-Trp-Val-Thr-Arg-Gln-(NMe)Arg-Tyr-NH₂

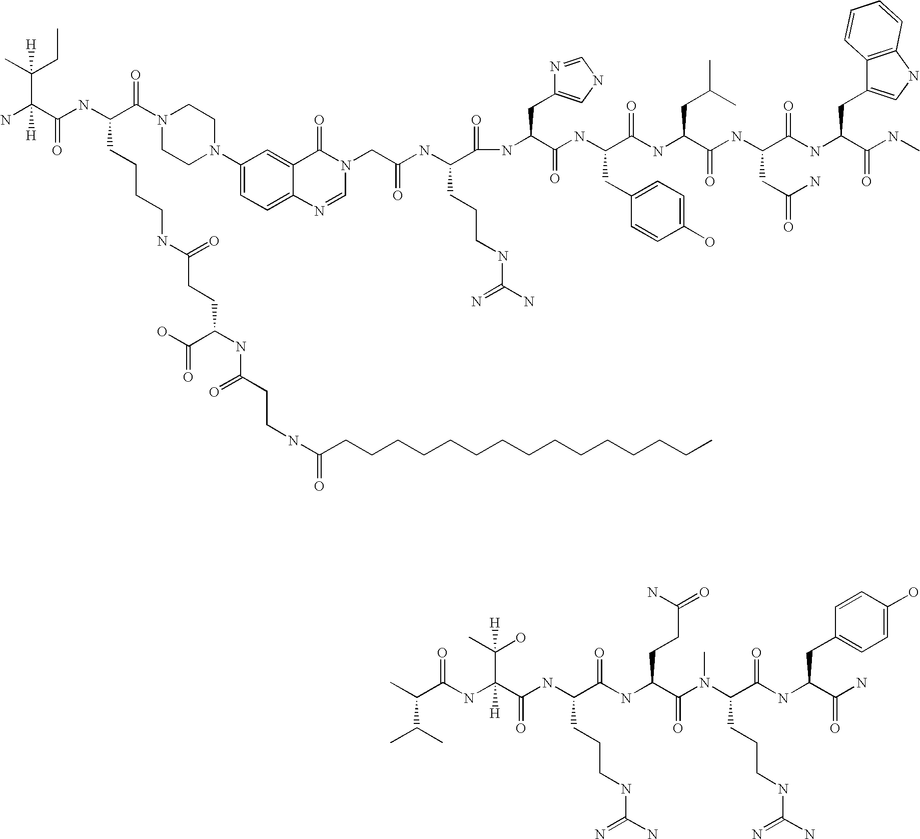

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-Glu-alphaOBu^{t} (426.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, Fmoc-beta-Ala (312.0 mg; 1.0 mmol), N-hydroxybenzotriazole (150 mg, 1.11 mmol), and N,N′-diisopropylcarbodiimide (1.50 mL, 2.0 mmol) were added and the reaction was allowed to proceed overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 34 mg (7%) of white amorphous powder. (ES)+-LCMS m/e calculated (calcd) C₁₂₈H₁₉₆N₃₆O₂₆ 2653.51, found 2653.50.

### Example 27

### Preparation of H-Ile-Lys(16-Bromohexadecanoyl-gamma-Glu-gamma-Glu-)-Pqa-Arg-His-Tyr-Leu-Asn-Trp-Val-Thr-Arg-Gln-(NMe)Arg-Tyr-NH₂

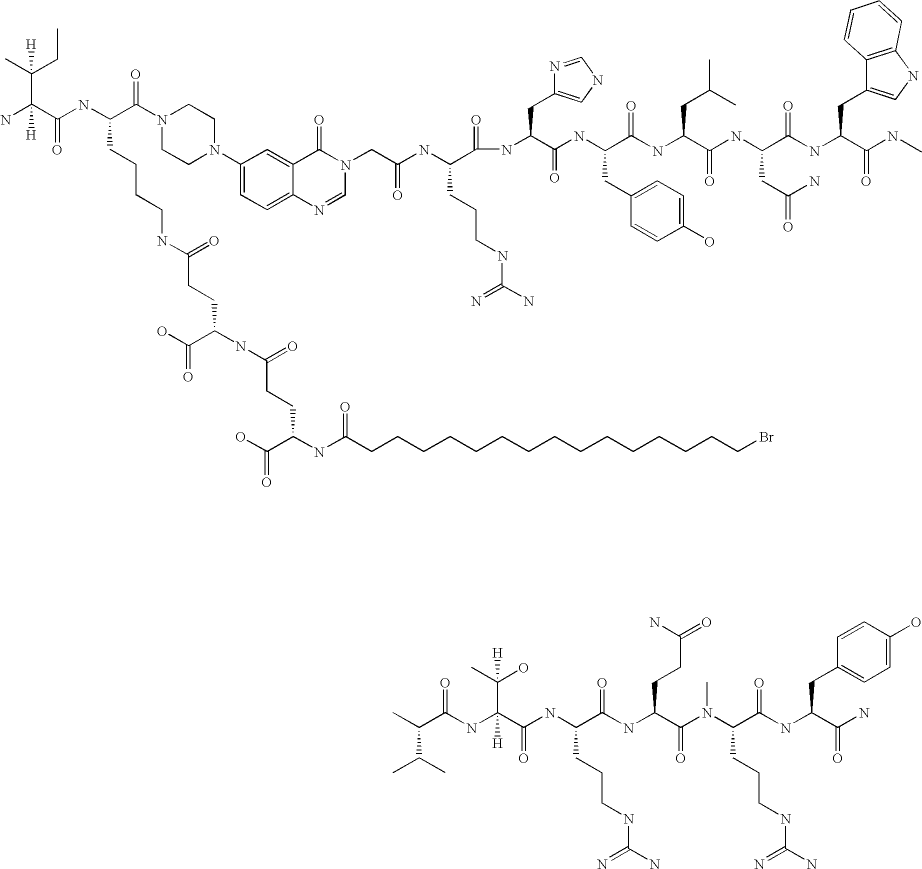

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-Glu-alphaOBut (426.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, Fmoc-Glu-alphaOBut (426.0 mg; 1.0 mmol), N-hydroxybenzotriazole (150 mg, 1.11 mmol), and N,N′-diisopropylcarbodiimide (1.50 mL, 2.0 mmol) were added and the reaction was allowed to proceed overnight. After Fmoc removal and washing with DMF, N-hydroxybenzotriazole (150 mg, 1.15 mmol), N,N′-diisopropylcarbodiimide (1.50 mL, 2.0 mmol) and 16-bromohexadecanoic acid (336 mg, 1.0 mmol) l were coupled overnight. After washing with DMF 2 times and CH₂Cl₂ 3 times, cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 61 mg (11%) of white amorphous powder. (ES)+-LCMS m/e calculated (calcd) C₁₃₀H₁₉₇BrN₃₆O₂₈ 2789.43, found 2789.41.

### Example 28

### Preparation of Pyro-Glu-Ile-Lys(Palmitoyl-gamma-Glu-gamma-Glu-)-Pqa-Arg-His-Tyr-Leu-Asn-Trp-Val-Thr-Arg-Gln-(NMe)Arg-Tyr-NH₂

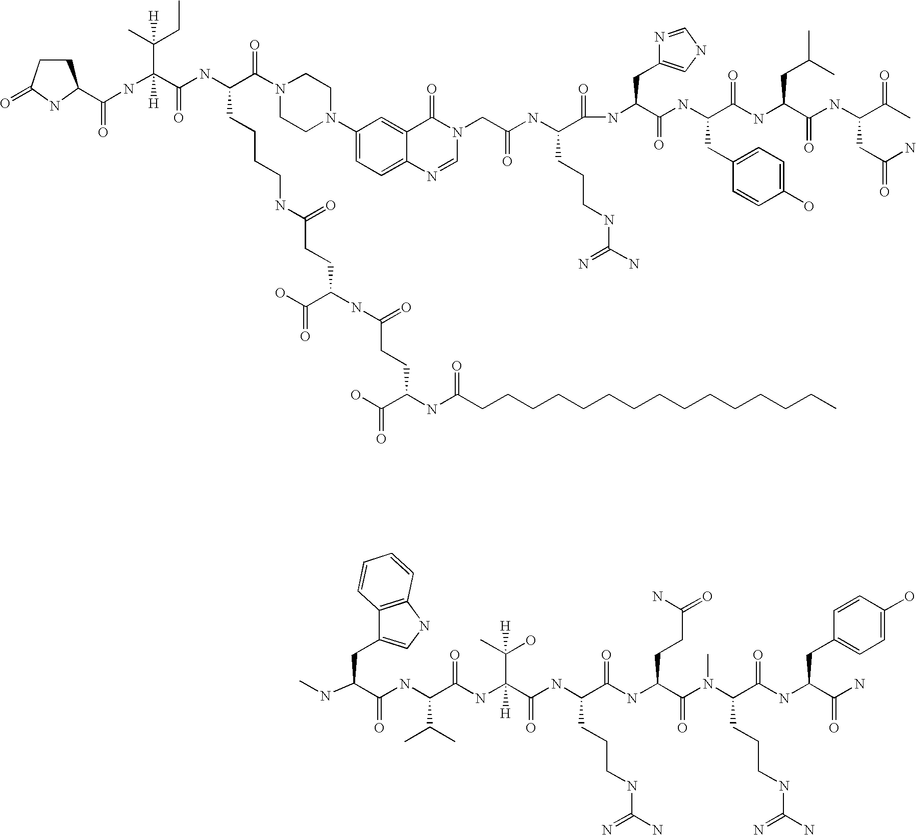

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in Example 8 and neutralization Fmoc-Glu-alphaOBut (426.0 mg; 1.0 mmol), N-hydroxybenzotriazole (150 mg, 1.11 mmol), and N,N′-diisopropylcarbodiimide (1.50 mL, 2.0 mmol) were coupled overnight. After Fmoc removal and washing with DMF, Fmoc-Glu-alphaOBut (426.0 mg; 1.0 mmol), N-hydroxybenzotriazole (150 mg, 1.11 mmol), and N,N′-diisopropylcarbodiimide (1.50 mL, 2.0 mmol) were coupled overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 58 mg (8.3%) of white amorphous powder. (ES)+-LCMS m/e calculated (calcd) C₁₃₅H₂₀₃N₃₇O₃₀ 2822.55, found 2822.55.

### Example 29

### Preparation of H-Ile-Lys(2-Hexadecanoyl-6Ahx)-Pqa-Arg-His-Tyr-Leu-Asn-Trp-Val-Thr-Arg-Gln-(NMe)Arg-Tyr-NH₂

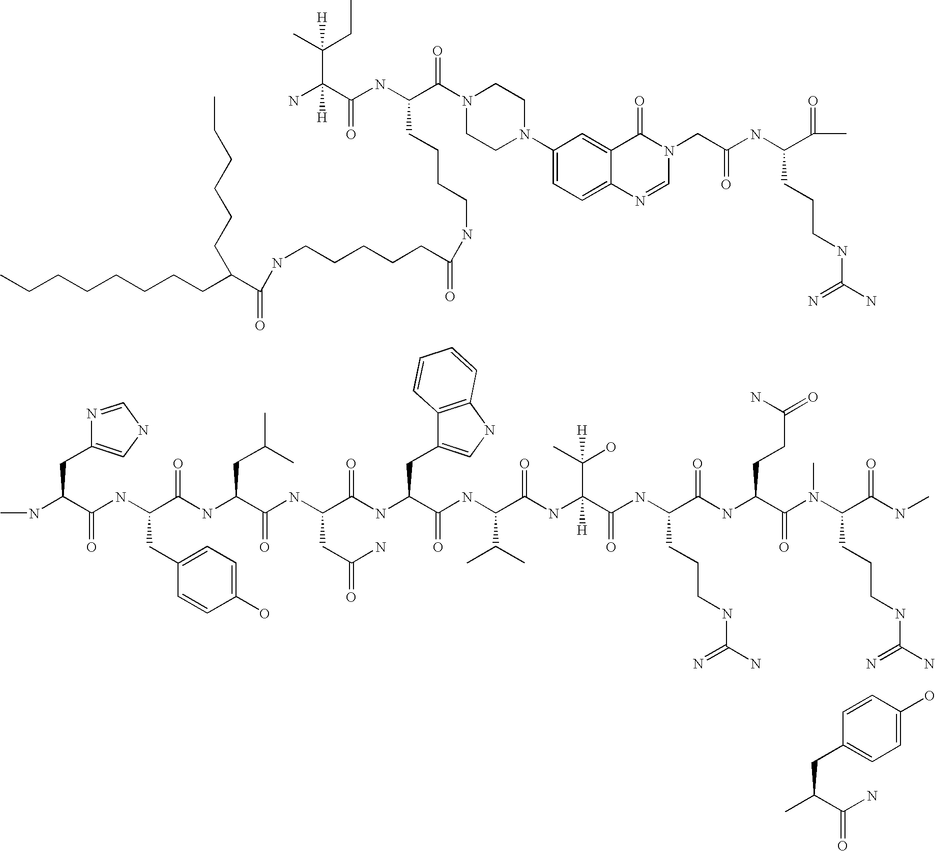

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (150 mg, 1.15 mmol), N,N′-diisopropylcarbodiimide (1.50 mL, 2.0 mmol) and 2-hexyldecanoic acid (286 mg, 1.0 mmole) were coupled overnight. After washing with DMF 2 times and CH₂Cl₂ 3 times, cleavage was effected with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 94 mg (18%) of white amorphous powder. (ES)+-LCMS m/e calculated (“calcd) C₁₂₆H₁₉₅N₃₅O₂₃ 2566.52, found 2566.51.

### Example 30

### Preparation of H-Ile-Lys(Eicosanoyl-6Ahx)-Pqa-Arg-His-Tyr-Leu-Asn-Trp-Val-Thr-Arg-Gln-(NMe)Arg-Tyr-NH₂

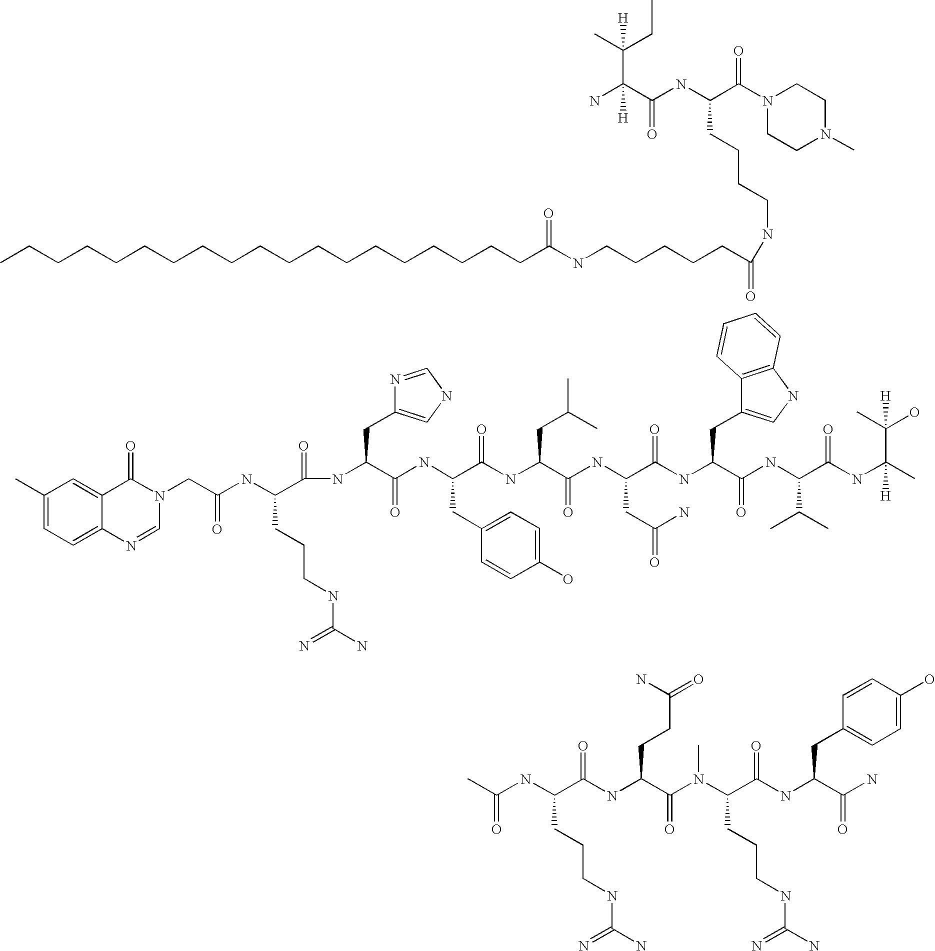

Boc-Ile-Lys(epsilon TFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-6-aminohexanoic acid (355.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, eicosanoic acid (315 mg, 1 mmol); N-hydroxybenzotriazole (150 mg, 1.11 mmol), and N,N′-diisopropylcarbodiimide (1.50 mL, 2.0 mmol) were coupled and stirred over night. After washing with DMF 2 times and CH₂Cl₂ 3 times cleavage with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 75 mg (14%) of white amorphous powder. (ES)+-LCMS m/e calculated (calcd) C₁₃₀H₂₀₃N₃₅O₂₃ 2622.58, found 2622.57.

### Example 31

### Preparation of H-Ile-Lys(Eicosanoyl-gamma-Glu-gamma-Glu-)-Pqa-Arg-His-Tyr-Leu-Asn-Trp-Val-Thr-Arg-Gln-(NMe)Arg-Tyr-NH₂

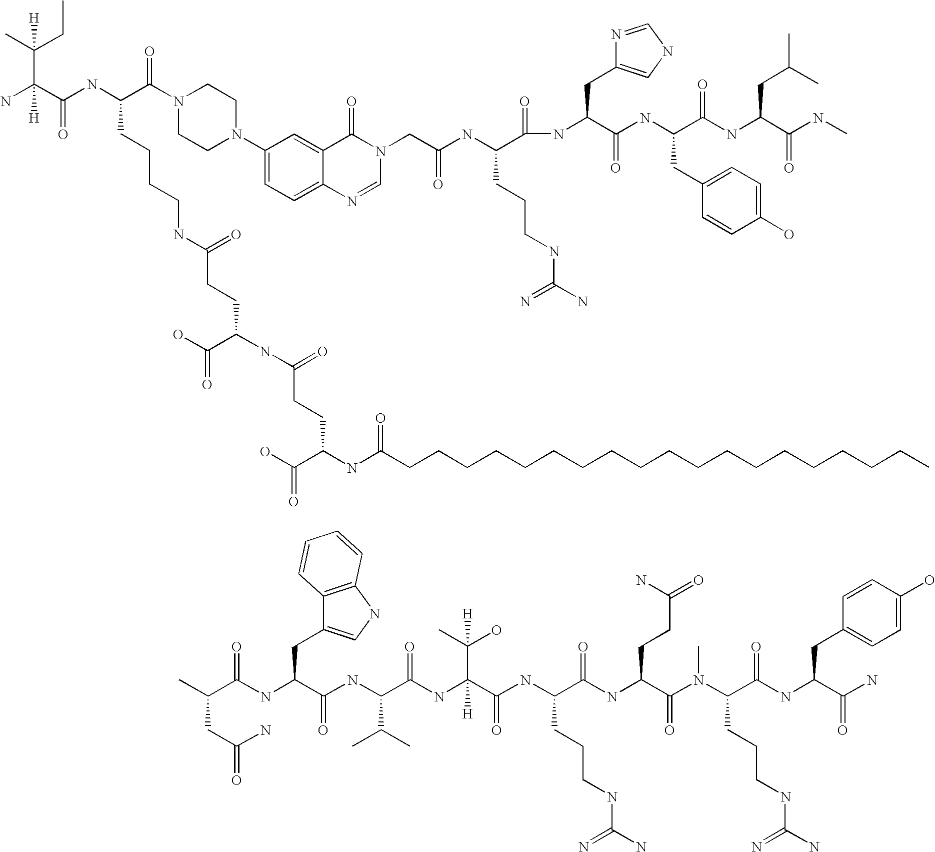

Boc-Ile-Lys(epsilonTFA salt)-Pqa-Arg(Pbf)-His(Trt)-Tyr(tBu)-Leu-Asn(Trt)-Trp-Val-Thr(tBu)-Arg(Pbf)-Gln(Trt)-NMe-Arg(Mtr)-Tyr(tBu)-Knorr resin 1.0 g was washed with 5% DIEA in DMF and coupled with Fmoc-Glu-alphaOBut (426.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, Fmoc-Glu-alpha OBu^{t} (426.0 mg; 1.0 mmol), N-hydroxybenzotriazole (150 mg, 1.11 mmol), and N,N′-diisopropylcarbodiimide (1.50 mL, 2.0 mmol) were added and the reaction allowed to proceed overnight. After Fmoc removal and washing with DMF, eicosanoic acid (315 mg, 1 mol); N-hydroxybenzotriazole (150 mg, 1.11 mmol), and N,N′-diisopropylcarbodiimide (1.50 mL, 2.0 mmol) were added to the resin bound peptide and the mixture was stirred over night. After washing with DMF 2 times and CH₂Cl₂ 3 times cleavage was effected with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 66 mg (12%) of white amorphous powder. (ES)+-LCMS m/e calculated (calcd) C₁₃₄H₂₀₆N₃₆O₂₈ 2767.58, found 2767.58.

### Example 32

### Preparation of H-Ile-Lys(Palmitoyl-15-ATOPA)-Pqa-Arg-His-Tyr-Leu-Asn-Trp-Val-Thr-Arg-Gln-(NMe)Arg-Tyr-NH₂

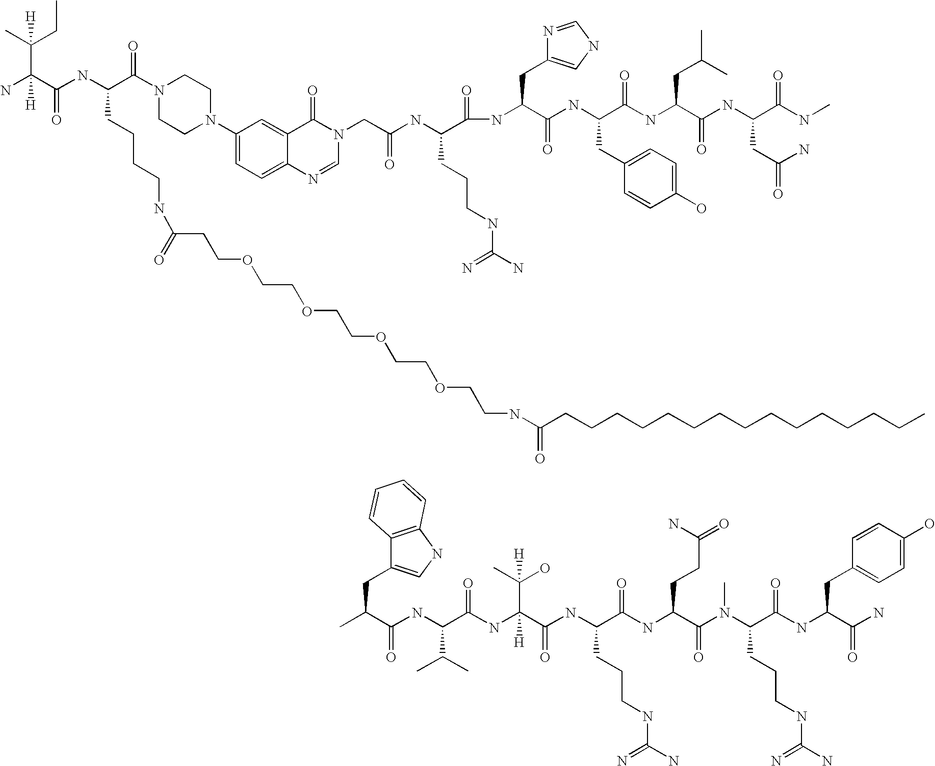

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in example 8 and neutralization, coupling with Fmoc-15-amino-4,7,10,13-tetraoxapentadecanoic acid (488 mg; 1.0 mm;), N-hydroxybenzotriazole (150 mg, 1.11 mmol), and N,N′-diisopropylcarbodiimide (1.50 mL, 2.0 mmol) was carried out overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 95 mg (14%) of white amorphous powder. (ES)+-LCMS m/e calculated (calcd) C₁₃₁H₂₀₅N₃₅O₂₇ 2700.57, found 2700.56.

### Example 33

### Preparation of H-Ile-Lys(Eicosanoyl-15-ATOPA)-Pqa-Arg-His-Tyr-Leu-Asn-Trp-Val-Thr-Arg-Gln-(NMe)Arg-Tyr-NH₂

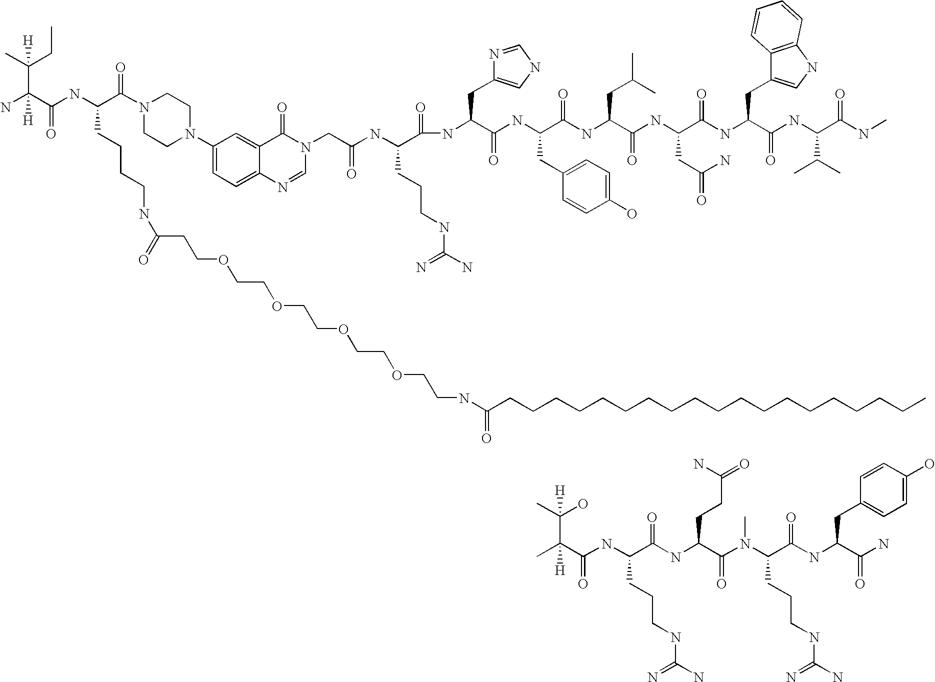

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in Example 8 and neutralization, coupling with Fmoc-15-amino-4,7,10,13-tetraoxapentadecanoic acid (488 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, eicosanoic acid (315 mg, 1 mmol); N-hydroxybenzotriazole (150 mg, 1.11 mmol), and N,N′-diisopropylcarbodiimide (1.50 mL, 2.0 mmol) were coupled and stirred over night. After washing with DMF 2 times and CH₂Cl₂ 3 times cleavage was effected with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 140 mg (20%) of white amorphous powder. (ES)+-LCMS m/e calculated (calcd) C₁₃₅H₂₁₃N₃₅O₂₇ 2756.64, found 2756.62.

### Example 34

### Preparation of H-Ile-Lys(Palmitoyl-12-ATODA)-Pqa-Arg-His-Tyr-Leu-Asn-Trp-Val-Thr-Arg-Gln-(NMe)Arg-Tyr-NH₂

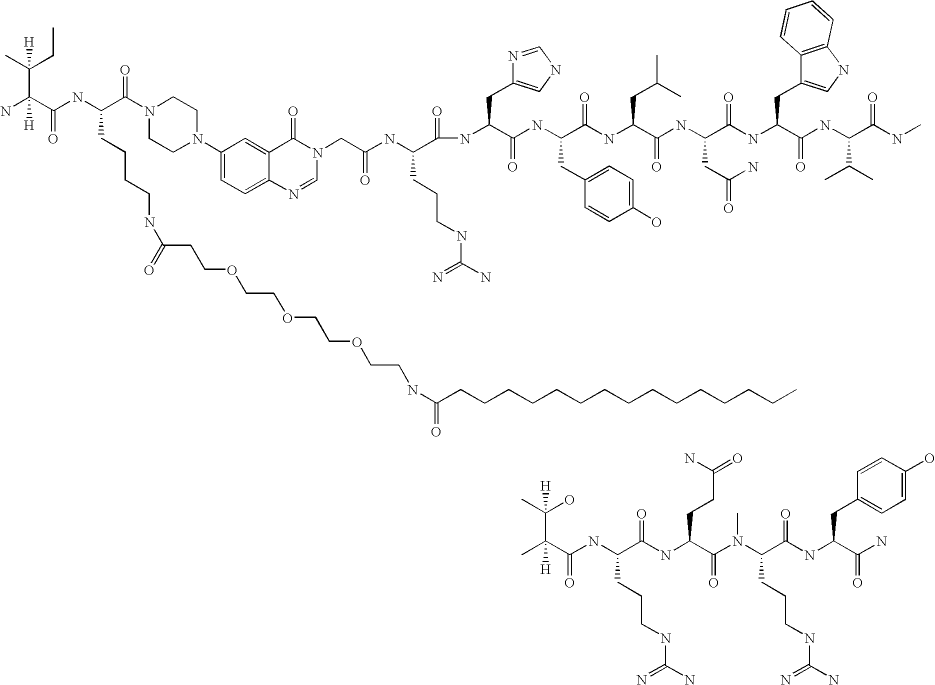

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in example 8 and neutralization, coupling with Fmoc-12-amino-4,7,10-trioxadodecanoic acid (488.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 134 mg (20%) of white amorphous powder. (ES)+-LCMS m/e calculated (calcd) C₁₂₉H₂₀₁N₃₅O₂₆ 2656.55, found 2656.54.

### Example 35

### Preparation of H-Ile-Lys(Eicosanoyl-12-ATODA)-Pqa-Arg-His-Tyr-Leu-Asn-Trp-Val-Thr-Arg-Gln-(NMe)Arg-Tyr-NH₂

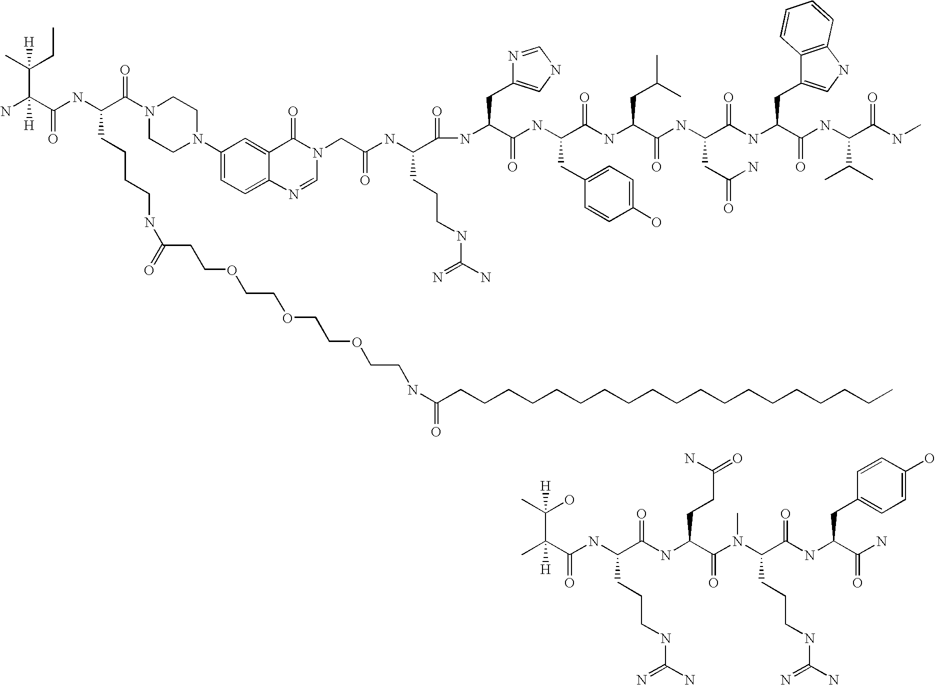

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in Example 8 and neutralization, coupling with Fmoc-12-Amino-4,7,10-trioxadodecanoic acid (488.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, eicosanoic acid (315 mg, 1 mmol); N-hydroxybenzotriazole (150 mg, 1.11 mmol), and N,N′-diisopropylcarbodiimide (1.50 mL, 2.0 mmol) were coupled and stirred over night. After washing with DMF 2 times and CH₂Cl₂ 3 times, cleavage was effected with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 128 mg (19%) of white amorphous powder. (ES)+-LCMS m/e calculated (calcd) C₁₃₃H₂₀₉N₃₅O₂₆ 2712.61, found 2712.59.

### Example 36

### Preparation of H-Ile-Lys(Palmitoyl-8-ADOSA)-Pqa-Arg-His-Tyr-Leu-Asn-Trp-Val-Thr-Arg-Gln-(NMe)Arg-Tyr-NH₂

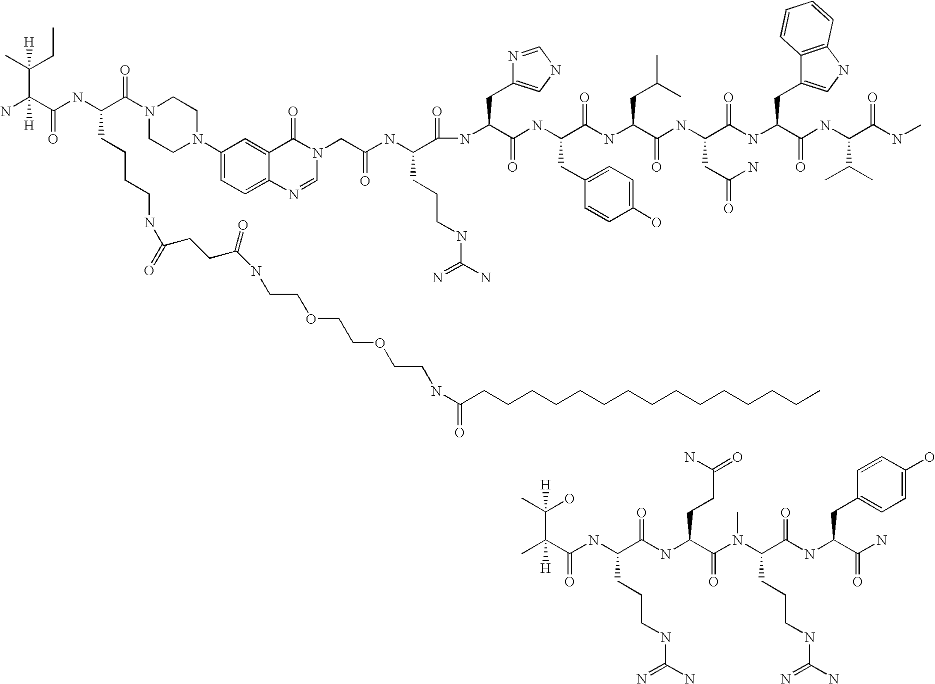

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection and neutralization, coupling with Fmoc-(8-Amino-3,6-dioxa-octyl)succinic acid (488.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 114 mg (17%) of white amorphous powder. (ES)+-LCMS m/e calculated (calcd) C₁₃₀H₂₀₂N₃₆O₂₆ 2683.56 found 2683.55.

### Example 37

### Preparation of H-Ile-Lys(Eicosanoyl-8-ADOSA)-Pqa-Arg-His-Tyr-Leu-Asn-Trp-Val-Thr-Arg-Gln-(NMe)Arg-Tyr-NH₂

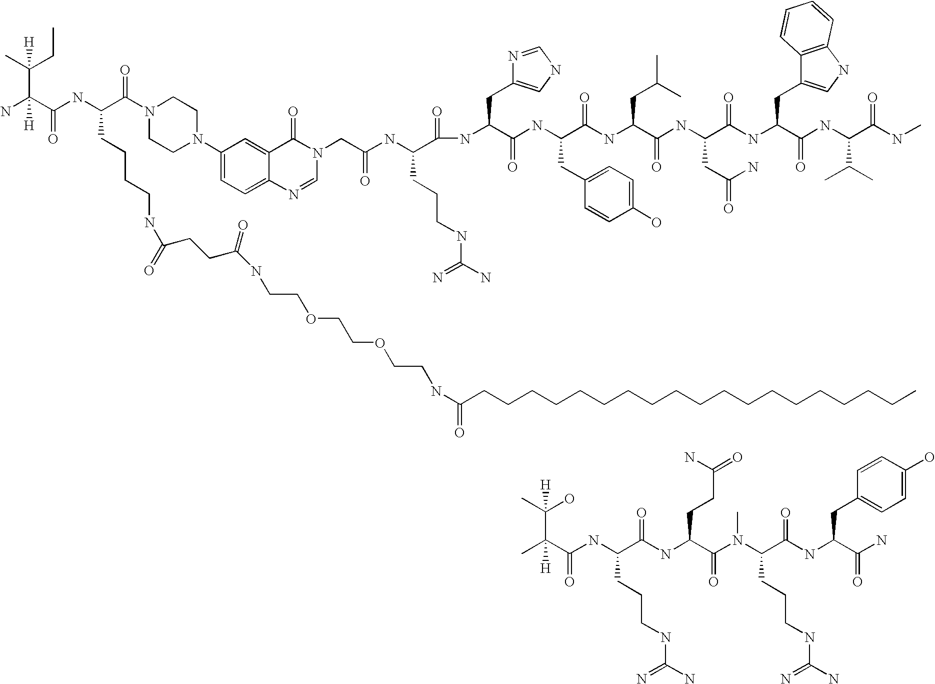

Fmoc-Linker-BHA resin (450 mg, 0.25 mmol) from Example 1 was subjected to solid phase synthesis with amine terminal Boc-Ile and Lys(alloc) in position for appropriate side chain modification. After palladium deprotection and neutralization, coupling with Fmoc-N-(8-amino-3,6-dioxa-octyl)succinamic acid (488.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, eicosanoic acid (315 mg, 1 mmol); N-hydroxybenzotriazole (150 mg, 1.11 mmol), and N,N′-diisopropylcarbodiimide (1.50 mL, 2.0 mmol) were coupled and stirred over night. After washing with DMF 2 times and CH₂Cl₂ 3 times, cleavage was effected with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 110 mg (16%) of white amorphous powder. (ES)+-LCMS m/e calculated (calcd) C₁₃₄H₂₁₀N₃₆O₂₆ 2739.62, found 2739.60.

### Example 38

### Preparation of H-Ile-Lys(Palmitoyl-5-AOPSA)-Pqa-Arg-His-Tyr-Leu-Asn-Trp-Val-Thr-Arg-Gln-(NMe)Arg-Tyr-NH₂

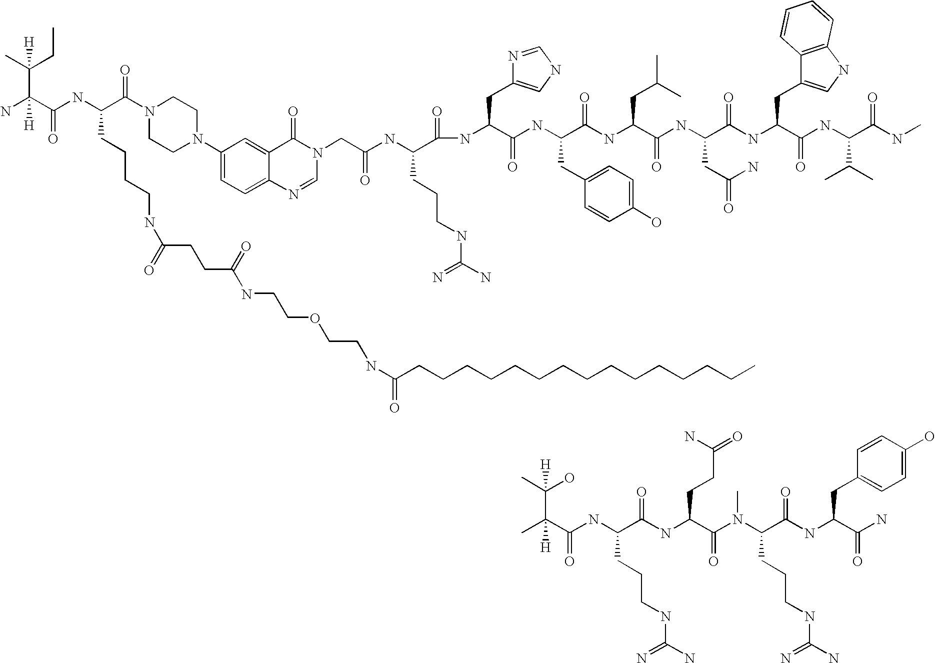

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in example 8 and neutralization, coupling was effected with N-Fmoc-(5-amino-3-oxa-pentyl)succinamic acid (427.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 158 mg (24%) of white amorphous powder. (ES)+-LCMS m/e calculated (calcd) C₁₂₈H₁₉₈N₃₆O₂₅ 2639.53, found 2639.50.

### Example 39

### Preparation of H-Ile-Lys(Eicosanoyl-5-AOPSA)-Pqa-Arg-His-Tyr-Leu-Asn-Trp-Val-Thr-Arg-Gln-(NMe)Arg-Tyr-NH₂

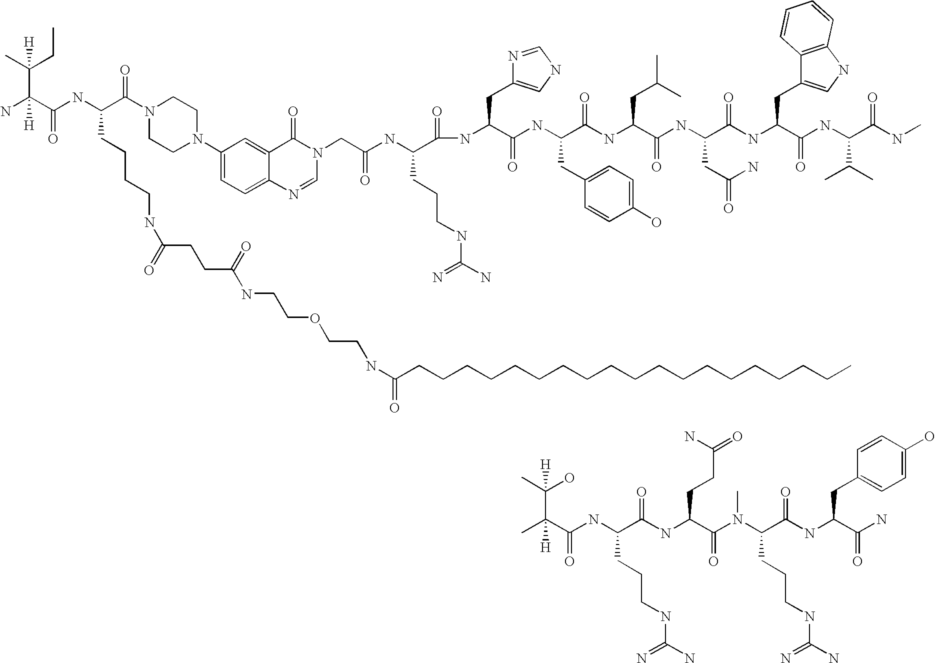

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in example 8 and neutralization, coupling with Fmoc-(5-amino-3-oxa-pentyl)succinamic acid (427.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF, eicosanoic acid (315 mg, 1 mol); N-hydroxybenzotriazole (150 mg, 1.11 mmol), and N,N′-diisopropylcarbodiimide (1.50 mL, 2.0 mmol) were added and the mixture was stirred over night. After washing with DMF 2 times and CH₂Cl₂ 3 times, cleavage was effected with TFA 17 mL, 400 uL iPrSiH and 800 uL propanethiol for 6 hr, the product was precipitated in 100.0 mL Et₂O, centrifuged, washed and dried in vacuo. The crude peptide was purified following the procedure in Example 7 to yield 128 mg (19%) of white amorphous powder. (ES)+-LCMS m/e calculated (calcd) C₁₃₂H₂₀₆N₃₆O₂₅ 2695.60, found 2695.59.

### Example 40

### Preparation of H-Ile-Lys(Palmitoyl-Ser-Ser)-Pqa-Arg-His-Tyr-Leu-Asn-Trp-Val-Thr-Arg-Gln-(NMe)Arg-Tyr-NH₂

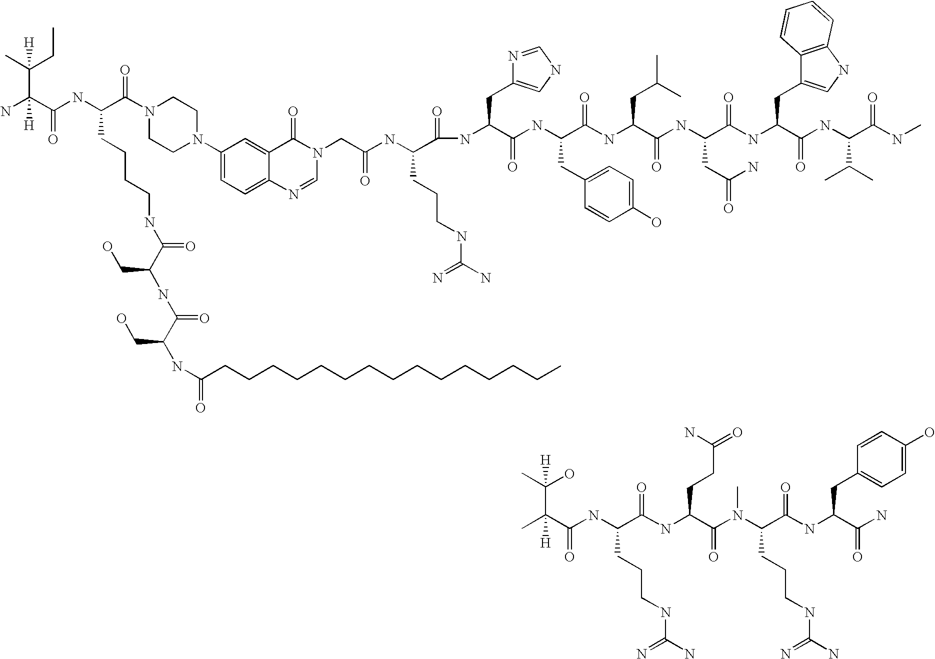

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection and neutralization, coupling with Fmoc-Ser(Bu^{t}) (384.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF the resin bound peptide was again coupled with Fmoc-Ser(Bu^{t}) (384.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 98 mg (15%) of white amorphous powder. (ES)+-LCMS m/e calculated (calcd) C₁₂₆H₁₉₄N₃₆O₂₆ 2627.50, found 2627.49.

### Example 41

### Preparation of H-Ile-Lys(Eicosanoyl-Ser-Ser)-Pqa-Arg-His-Tyr-Leu-Asn-Trp-Val-Thr-Arg-Gln-(NMe)Arg-Tyr-NH₂

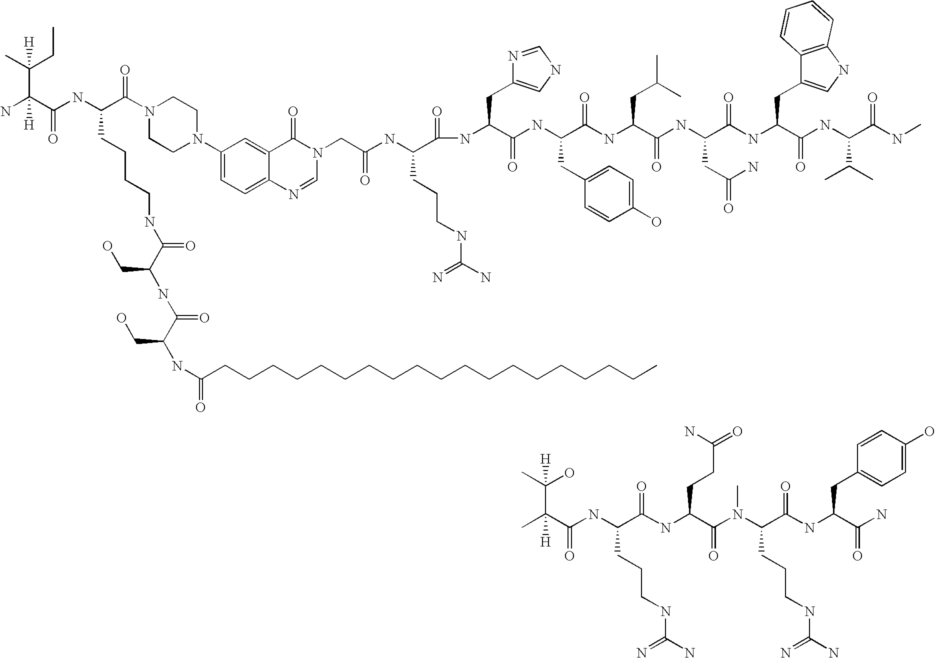

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in Example 8 and neutralization, coupling with Fmoc-Ser(Bu^{t}) (384.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF the resin was again coupled with Fmoc-Ser(Bu^{t}) (384.0 mg; 1.0 mmol), N-hydroxybenzotriazole (150 mg, 1.11 mmol), and N,N′-diisopropylcarbodiimide (1.50 mL, 2.0 mmol) overnight. Eicosanoic acid (315 mg, 1 mol); N-hydroxybenzotriazole (150 mg, 1.11 mmol), and N,N′-diisopropylcarbodiimide (1.50 mL, 2.0 mmol) were coupled to the resin bound peptide overnight. After washing with DMF 2 times and CH₂Cl₂ 3 times, cleavage was effected with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 107 mg (16%) of white amorphous powder. (ES)+-LCMS m/e calculated (calcd) C₁₃₀H₂₀₂N₃₆O₂₆ 2683.56, found 2683.55.

### Example 42

### Preparation of H-Ile-Lys(Palmitoyl-Thr-Thr)-Pqa-Arg-His-Tyr-Leu-Asn-Trp-Val-Thr-Arg-Gln-(NMe)Arg-Tyr-NH₂

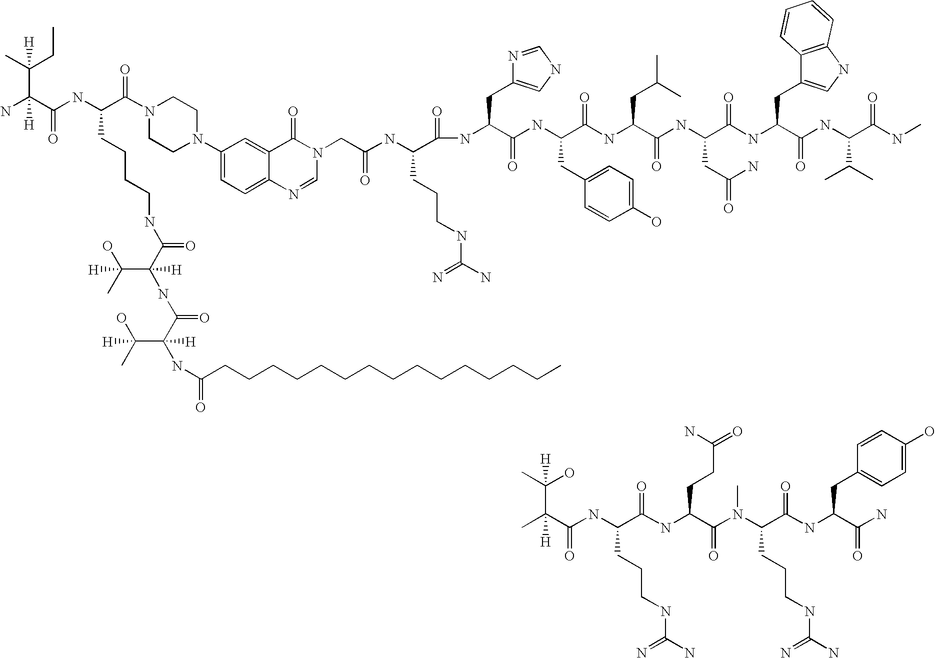

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in Example 8 and neutralization, coupling with Fmoc-Thr(Bu^{t}) (398.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF the resin was again coupled with Fmoc-Thr(Bu^{t}) (398.0 mg; 1.0 mmol), N-hydroxybenzotriazole (150 mg, 1.11 mmol), and N,N′-diisopropylcarbodiimide (1.50 mL, 2.0 mmol) overnight. After Fmoc removal and washing with DMF, N-hydroxybenzotriazole (425 mg, 3.150 mmol), DIEA (500 uL, 3.0 m) and palmitoyl chloride (2.8 mL, 2.75 m) were reacted in 15 mL CH₂Cl₂ for 5 min and added to the peptide resin. The reaction mixture was stirred over night and washed with DMF 2 times and CH₂Cl₂ 3 times before cleavage was effected with TFA, 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 73 mg (11%) of white amorphous powder. (ES)+-LCMS m/e calculated (calcd) C₁₂₈H₁₉₈N₃₆O₂₆ 2655.53, found 2655.51.

### Example 43

### Preparation of H-Ile-Lys(Eicosanoyl-Thr-Thr)-Pqa-Arg-His-Tyr-Leu-Asn-Trp-Val-Thr-Arg-Gln-(NMe)Arg-Tyr-NH₂

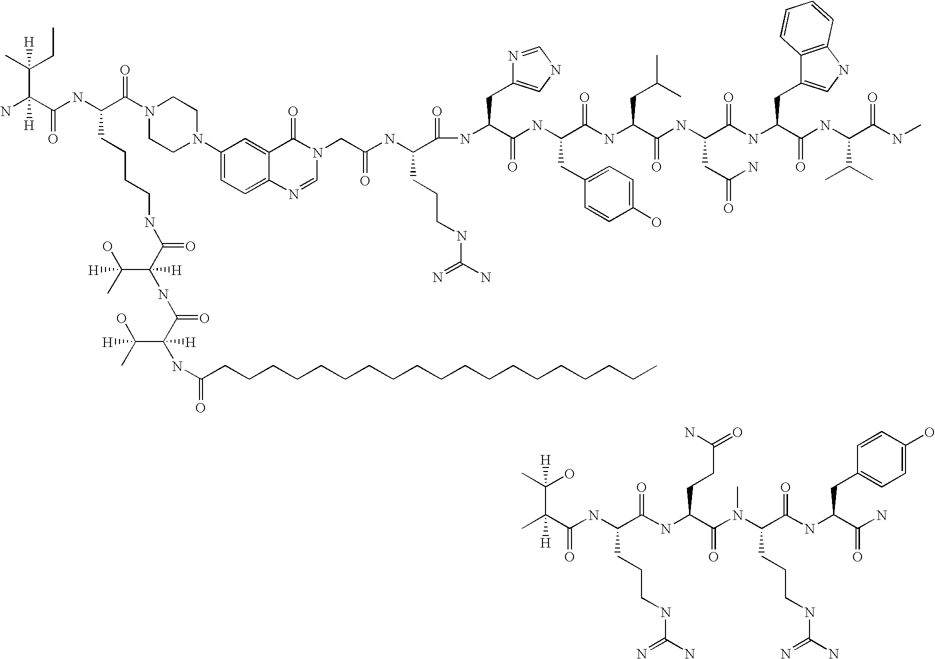

Fmoc-Linker-BHA resin (450 mg, 0.25 mmol) from Example 1 was subjected to solid phase synthesis with amine terminal Boc-Ile and Lys(alloc) in position for appropriate side chain modification. After palladium catalyzed deprotection as described in Example 8 and neutralization, coupling with Fmoc-Thr(Bu^{t}) (398.0 mg; 1.0 mmol), N-hydroxybenzotriazole (150 mg, 1.11 mmol), and N,N′-diisopropylcarbodiimide (1.50 mL, 2.0 mmol) was carried out overnight. After Fmoc removal and washing with DMF the resin was again coupled with Fmoc-Thr(Bu^{t}) (398.0 mg; 1.0 mmol), N-hydroxybenzotriazole (150 mg, 1.11 mmol), and N,N′-diisopropylcarbodiimide (1.50 mL, 2.0 mmol) was carried out overnight. Eicosanoic acid (315 mg, 1 mmol); N-hydroxybenzotriazole (150 mg, 1.11 mmol), and N,N′-diisopropylcarbodiimide (1.50 mL, 2.0 mmol) were added and the mixture was stirred over night. After washing with DMF 2 times and CH₂Cl₂ 3 times cleavage was effected with TFA 17 mL, 400 uL iPrSiH and 800 uL propanethiol for 6 hr. The product was precipitated in 100.0 mL Et₂O, centrifuged, washed and dried in vacuo. The crude peptide was purified following the procedure in Example 7 to yield 69 mg (10%) of white amorphous powder. (ES)+-LCMS m/e calculated (calcd) C₁₃₂H₂₀₆N₃₆O₂₆ 2711.59, found 2711.57

### Example 44

cAMP Agonist Assay

In this example, the following materials were used: 384-well plate; Tropix cAMP-Screen Kit; cAMP ELISA System (Applied Biosystems, cat. #T1505; CS 20000); Forskolin (Calbiochem cat. #344270); cells: HEK293/hNPY2R; growth medium: Dulbecco's modified eagle medium (D-MEM, Gibco); 10% Fetal bovine serum (FBS, Gibco), heat-inactivated; 1% Penicillin/Streptomycin (Pen 10000 unit/mL: Strep 10000 mg/mL, Gibco); 500 mg/mL G418 (Geneticin, Gibco cat. #11811-031); and plating medium: DMEM/F12 w/o phenol red (Gibco); 10% FBS (Gibco, cat. #10082-147), heat-inactivated; 1% Penicillin/Streptomycin (Gibco, cat. #15140-122); 500 mg/mL G418 (Geneticin, Gibco, cat. #11811-031).

On the first day, medium was discarded, and the monolayer cells were washed with 10 mL PBS per flask (T225). After decanting with PBS, 5 mL VERSENE (Gibco, cat #1504006) was used to dislodge the cells (5 min @37 C). The flask was gently tapped and the cell suspension was pooled. Each flask was rinsed with 10 mL plating medium and centrifuged at 1000 rpm for 5 min. The suspension was pooled and counted. The suspension was resuspended in plating medium at a density of 2.0×10⁵ cells/mL for HEK293/hNPY2R. 50 microliters of cells (HEK293/hNPY2R-10,000 cells/well) were transferred into the 384-well plate using Multi-drop dispenser. The plates were incubated at 37° C. overnight. On the second day, the cells were checked for 75-85% confluence. The media and reagents were allowed to come to room temperature. Before the dilutions were prepared, the stock solution of stimulating compound in dimethyl sulphoxide (DMSO, Sigma, cat #D2650) was allowed to warm up to 32 C for 5-10 min. The dilutions were prepared in DMEM/F12 with 0.5 mM 3-Isobutyl-1-methylxanthine (IBMX, Calbiochem, cat #410957) and 0.5 mg/mL BSA. The final DMSO concentration in the stimulation medium was 1.1% with Forskolin concentration of 5 μM. The cell medium was tapped off with a gentle inversion of the cell plate on a paper towel. 50 μL of stimulation medium was placed per well (each concentration done in four replicates). The plates were incubated at room temperature for 30 min, and the cells were checked under a microscope for toxicity. After 30 minutes of treatment, the stimulation media was discarded and 50 mL/well of Assay Lysis Buffer (provided in the Tropix kit) was added. The plates were incubated for 45 min@37° C. 20 μL of the lysate was transferred from stimulation plates into the pre-coated antibody plates (384-well) from the Tropix kit. 10 μL of AP conjugate and 20 μL of anti-cAMP antibody was added. The plates were incubated at room temperature while shaking for 1 hour. The plates were then washed 5 times with Wash Buffer, 70 μL per well for each wash. The plates were tapped to dry. 30 μL/well of CSPD/Saphire-II RTU substrate/enhancer solution was added and incubated for 45 min @ RT (shake). Signal for 1 sec/well in a Luminometer. (VICTOR-V) was measured.

### Example 45

### CaFlux Assay

Hek-293 cells were stably transfected with the G protein chimera Gaqi9 and the hygromycin-B resistance gene were further transfected with the human NPY2 receptor and G418 antibiotic selection. Following selection in both hygromycin-B and G418, individual clones were assayed for their response to PYY. The transfected cells were cultured in DMEM medium supplemented with 10% fetal bovine serum, 50 μg/mL hygromycin-B 2 mM glutamine, 100 U/mL penicillin, 100 μg/mL streptomycin and 250 μg/mL G418. Cells are harvested with trypsin-EDTA and counted using ViaCount reagent. The cell suspension volume is adjusted to 4.8×10⁵ cells/mL with complete growth media. Aliquots of 25 μL are dispensed into 384 well Poly-D Lysine coated black/clear microplates (Falcon) and the microplates were placed in a 37° C. CO₂ incubator overnight. Loading Buffer (Calcium-3 Assay Kit, Molecular Devices) was prepared by dissolving the contents of one vial (Express Kit) into 1000 mL Hank's Balanced Salt Solution containing 20 mM HEPES and 5 mM probenecid. Aliquots of 25 μL of diluted dye were dispensed into the cell plates and the plates are then incubated for 1 hour at 37° C. During the incubation, test compounds were prepared at 3.5× the desired concentration in HBSS (20 mM HEPES)/0.05% BSA/1% DMSO and transferred to a 384 well plate for use on FLIPR. After incubation, both the cell and compound plates were brought to the FLIPR and 20 μL of the diluted compounds were transferred to the cell plates by the FLIPR. During the assay, fluorescence readings were taken simultaneously from all 384 wells of the cell plate every 1.5 seconds. Five readings were taken to establish a stable baseline, and then 20 μL of sample was rapidly (30 μL/sec) and simultaneously added to each well of the cell plate. The fluorescence was continuously monitored before, during and after sample addition for a total elapsed time of 100 seconds. Responses (increase in peak fluorescence) in each well following addition were determined. The initial fluorescence reading from each well, prior to ligand stimulation, was used as a zero baseline value for the data from that well. The responses are expressed as % of maximal response of the positive control.

The compounds of the present invention exhibited selective Neuropeptide-2 receptor activity in vitro, as demonstrated in the cAMP assay and CaFlux Assay (FLIPR). Summary of the in vitro results, 1050 and EC50 for representative compounds of the invention, are illustrated in Table 1 below:

TABLE 1

| | | | | | | |
|---|---|---|---|---|---|---|
| | | Y2R | Y2R | Y1R | Y4R | Y5R |
| | | EC50 | EC50 | EC50 | EC50 | EC₅₀ |
| | | (nM) | (nM) | (nM) | (nM) | (nM) |
| Exaple | Sequence | FLIRR | cAMP | FLIPR | FLIPR | FLIPR |

| | | | | | | |
|---|---|---|---|---|---|---|
| 1 | Fmoc-linker-BHA-Resin | | | | | |
| 2 | ABI-protocol | | | | | |
| 3 | IKPEAPGEDASPEELNRYYASLRHY | 0.013 | 0.038 | 356 | 1187 | 121 |
| | LNLVTRQRY (PYY 3-36) | | | | | |
| 4 | Ac-IK-Pqa-RHYLNWVTRQ(N- | 0.21 | 0.34 | >5000 | >5000 | >5000 |
| | methyl)RY | | | | | |
| 5 | Ac-IK(Butyryl)-Pqa- | 0.18 | 0.39 | >5000 | 31633 | 24896 |
| | RHYLNWVTRQ(N-methyl)RY | | | | | |
| 6 | Ac-IK(Capryloyl)-Pqa- | 1.45 | 1.7 | 5200 | 2467 | 99894 |
| | RHYLNWVTRQ(N-methyl)RY | | | | | |
| 7 | Ac-IK(Lauroyl)-Pqa- | 4.7 | 5.4 | 6433 | 14467 | 12845 |
| | RHYLNWVTRQ(N-methyl)RY | | | | | |
| 8 | Protected Peptide Resin | | | | | |
| 9 | IK(Lauroyl-6Ahx)-Pqa- | 0.031 | 3.5 | >5000 | 2449 | 3793 |
| | RHYLNWVTRQ(N-methyl)RY | | | | | |
| 10 | IK(Lauroyl-heta-Ala)-Pqa- | 0.016 | 5.2 | >5000 | 3507 | 4743 |
| | RHYLNWVTRQ(N-methyl)RY | | | | | |
| 11 | IK(Lauroyl-Glu)-Pqa- | 0.026 | 3.6 | >5000 | 2427 | 3554 |
| | RHYLNWVTRQ(N-methyl)RY | | | | | |
| 12 | IK(Myrisoyl-6Ahx)-Pqa- | 0.14 | 0.16 | >5000 | >5000 | 1422 |
| | RHYLNWVTRQ(N-methyl)RY | | | | | |
| 13 | Ac-IK(Palmitoyl)-Pqa- | 1.31 | 1.2 | 29233 | 32167 | 9379 |
| | RHYLNWVTRQ(N-methyl)RY | | | | | |
| 14 | IK(Palmitoyl)-Pqa- | 0.73 | 1 | >5000 | >5000 | 12666 |
| | RHYLNWVTRQ(N-methyl)RY | | | | | |
| 15 | Palmitoyl-IK-Pqa- | 1.03 | 0.97 | >5000 | 1355 | >5000 |
| | RHYLNWVTRQ(N-methyl)RY | | | | | |
| 16 | Palmitoyl-6Ahx-IK-Pqa- | 0.18 | 0.23 | >5000 | 13700 | 544 |
| | RHYLNWVTRQ(N-methyl)RY | | | | | |
| 17 | Palmitoyl-6Ahx-IK-Pqa- | 0.09 | 0.25 | >5000 | 14500 | 27 |
| | RHYLNWVTRQRY | | | | | |
| 18 | IK(Palmitoyl-6Ahx)-Pqa- | 0.012 | 0.18 | >5000 | >5000 | 1185 |
| | RHYLNWVTRQ(N-methyl)RY | | | | | |
| 19 | IK(Palmitoyl-6Ahx)-Pqa- | 0.004 | 0.15 | >5000 | >5000 | 45 |
| | RHYLNWVTRQRY | | | | | |
| 20 | IK(Palmitoyl-beta Ala)-Pqa- | 0.015 | 0.26 | >5000 | >5000 | 1878 |
| | RHYLNWVTRQ(N-methyl)RY | | | | | |
| 21 | IK(Palmitoyl-Glu)-Pqa- | 0.43 | 1 | >5000 | >5000 | 4185 |
| | RHYLNWVTRQ(N-methyl)RY | | | | | |
| 22 | IK(Palmitoyl-heta Ala-Glu)-Pqa- | 0.048 | 0.15 | >5000 | >5000 | 227 |
| | RHYLNWVTRQ(N-methyl)RY | | | | | (70%) |
| 23 | IK(Palmitoyl-Glu-Glu)-Pqa- | 0.033 | 0.29 | >5000 | >5000 | 459 |
| | RHYLNWVTRQ(N-methyl)RY | | | | | (70%) |
| 24 | IK(Palmitoyl-gamaGlu)-Pqa- | 0.039 | 0.21 | >5000 | >5000 | 168 |
| | RHYLNWVTRQ(N-methyl)RY | | | | | (70%) |
| 25 | IK(Palmitoyl-gamaGlu-gamaGlu)-Pqa- | 0.08 | 0.22 | >5000 | >5000 | 443 |
| | RHYLNWVTRQ(N-methyl)RY | | | | | (70%) |
| 26 | IK(Palmitoyl-heta Ala-gamaGlu)-Pqa- | 0.045 | 0.15 | >5000 | >5000 | 129 |
| | RHYLNWVTRQ(N-methyl)RY | | | | | (70%) |
| 27 | IK(16-Bromohexadecanoyl-gamaGlu- | 0,23 | 0.4 | >5000 | >5000 | 2536 |
| | gamaGlu)-Pqa-RHYLNWVTRQ(N- | | | | | |
| | methyl)RY | | | | | |
| 28 | PyroGlu-IK(Palmitoyl-gamaGlu- | 0.176 | 0.21 | >5000 | >5000 | 2062 |
| | gamaGlu)-Pqa-RHYLNWVTRQ(N- | | | | | |
| | methyl)RY | | | | | |
| 29 | IK(2-hexyldecanoyl-6Ahx)-Pqa- | 0.361 | 2.8 | >5000 | >5000 | >5000 |
| | RHYLNWVTRQ(N-methyl)RY | | | | | |
| 30 | IK(Eicosanoyl-6Ahx)-Pqa- | 0.96 | 0.14 | >5000 | >5000 | 306 |
| | RHYLNWVTRQ(N-methyl)RY | | | | | (28%) |
| 31 | IK(Eicosanoyl-gamaGlu-gamaGlu)- | 0.091 | 0.07 | >5000 | >5000 | 634 |
| | Pga-RHYLNWVTRQ(N-methyl)RY | | | | | (60%) |
| 32 | IK(Palmitoyl-15-ATOPA)-Pqa- | 0.26 | 0.19 | >5000 | >5000 | 973 |
| | RHYLNWVTRQ(N-methyl)RY | | | | | (28%) |
| 33 | IK(Eicosanoyl-15-ATOPA)-Pqa- | 1.08 | 0.13 | >5000 | >5000 | 241 |
| | RHYLNWVTRQ(N-methyl)RY | | | | | (53%) |
| 34 | IK(Palmitoyl-12-ATODA)-Pqa- | 0.003 | 0.1 | >5000 | >5000 | 2337 |
| | RHYLNWVTRQ(N-methyl)RY | | | | | (80%) |
| 35 | IK(Eicosanoyl-12-ATODA)-Pqa- | 1.02 | 0.11 | >5000 | >5000 | 501 |
| | RHYLNWVTRQ(N-methyl)RY | | | | | (65%) |
| 36 | IK(Palmitoyl-8-ADOSA)-Pqa- | 0.138 | 0.15 >5000 | >5000 | 481 | |
| | RHYLNWVTRQ(N-methyl)RY | | | | | (73%) |
| 37 | IK(Eicosanoyl-8-ADOSA)-Pqa- | 0.367 | 0.13 | >5000 | >5000 | 77.9 |
| | RHYLNWVTRQ(N-methyl)RY | | | | | (48%) |
| 38 | IK(Palmitoyl-5-APOSA)-Pqa- | 0.003 | 0.17 | >5000 | >5000 | 644 |
| | RHYLNWVTRQ(N-methyl)RY | | | | | (83%) |
| 39 | IK(Eicosanyl-5-APOSA)-Pqa- | 0.073 | 0.21 | >5000 | >5000 | 285 |
| | RHYLNWVTRQ(N-methyl)RY | | | | | (50%) |
| 40 | IK(Palmitoyl-Ser-Ser)-Pqa- | 0.36 | 0.18 | >5000 | >5000 | 602 |
| | RHYLNWVTRQ(N-methyl)RY | | | | | (85%) |
| 41 | IK(Eicosanoyl-Ser-Ser)-Pqa- | 0.165 | 0.11 | >5000 | >5000 | 1833 |
| | RHYLNWVTRQ(N-methyl)RY | | | | | (37%) |
| 42 | IK(Palmitoyl-Thr-Thr)-Pqa- | 0.018 | 0.14 | >5000 | >5000 | 193 |
| | RHYLNWVTRQ(N-methyl)RY | | | | | (46%) |
| 43 | IK(Eicosanoyl-Thr-Thr)-Pqa- | 0.074 | 0.26 | >5000 | >5000 | 243 |
| | RHYLNWVTRQ(N-methyl)RY | | | | | (23%) |

It is to be understood that the invention is not limited to the particular embodiments of the invention described above, as variations of the particular embodiments may be made and still fall within the scope of the appended claims.

What is claimed is:

1. A neuropeptide-2 receptor agonist of the formula (I): 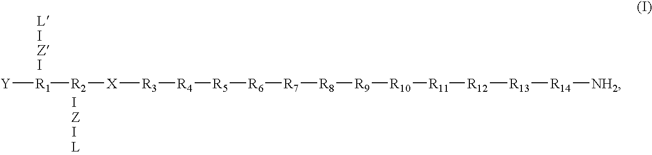
wherein:
L, L′ is a lipid moiety;
X is (4-oxo-6-piperazin-1-yl-4H-quinazolin-3-yl)-acetic acid (Pqa);
Y is H, an acyl moiety or gyro-Glu;
Z, Z′ is a spacer moiety or absent;
R₁ is Ile, Ala, (D)Ile or N-methyl Ile;
R₂ is Lys, Ala, (D)Lys, N-methyl lys, Nle or (Lys-Gly);
R₃ is Arg, Ala, (D)Arg, N-methyl Arg or Phe;
R₄ is His, Ala, (D)His or N-methyl His;
R₅ is Tyr, Ala, (D)Tyr, N-methyl Tyr or Trp;
R₆ is Leu, Ala, (D)Leu or N-methyl Leu;
R₇ is Asn, Ala or (D)Asn;
R₈ is Leu or Trp;
R₉ is Val, Ala, (D)Val or N-methyl Val;
R₁₀ is Thr, Ala or N-methyl Thr;
R₁₁ is Arg, (D)Arg or N-methyl Arg;
R₁₂ is Gln or Ala;
R₁₃ is Arg, (D)Arg or N-methyl Arg; and
R₁₄ is Tyr, (D) Tyr, N-methyl Tyr, Phe or Tip,
wherein moieties L-Z- and L′-Z′- are not both present,
or a pharmaceutically acceptable salt thereof.

2. The neuropeptide-2 receptor agonist according to claim 1, wherein said lipid moiety is carpryloyl, lauroyl, myristoyl, palmitoyl, 16-bromohexadecanoyl, 2-hexyldecanoyl or eicosanoyl.

3. The neuropeptide-2 receptor agonist according to claim 1, wherein said spacer moiety is Ahx, Ala, Glu, Ala-Glu, Glu-Glu, ATOPA, ATODA, ADOSA, AOPSA, Ser-Ser or Thr-Thr.

4. The neuropeptide-2 receptor agonist according to claim 1, wherein Z is absent.

5. The neuropeptide-2 receptor agonist according to claim 1, wherein Z′ is absent.

6. The neuropeptide-2 receptor agonist according to claim 1, having formula (II): 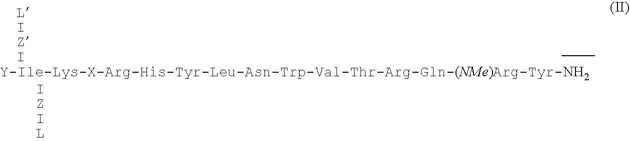
wherein:
L, L′ is a lipid moiety;
X is (4-oxo-6-piperazin-1-yl-4H-quinazolin-3-yl)-acetic acid (Pqa);
Y is H, an acyl moiety or gyro-Glu; and
Z, Z′ is Ahx, Ala, Glu, Ala-Glu, Glu-Glu, ATOPA, ATODA, ADOSA, AOPSA, Ser-Ser Thr-Thr- or absent,
wherein moieties L-Z- and L′-Z′- are not both present.

7. The neuropeptide-2 receptor agonist according to claim 6, wherein said lipid moiety is carpryloyl, lauroyl, myrisoyl, palmitoyl, 16-bromohexadecanoyl, 2-hexyldecanoyl or eicosanoyl.

8. The neuropeptide-2 receptor agonist according to claim 6, wherein Z, Z′ is Ala, Glu, Ala-Glu, Glu-Glu, Ser-Ser or Thr-Thr

9. The neuropeptide-2 receptor agonist according to claim 6, wherein Z is absent.

10. The neuropeptide-2 receptor agonist according to claim 6, wherein Z′ is absent.

11. The neuropeptide-2 receptor agonist according to claim 1, selected from the group consisting of:
Ac-Ile-Lys(Butyryl)-Pqa-Arg-His-Tyr-Leu-Asn-Trp-
Val-Thr-Arg-Gln-(NMe)Arg-Tyr-NH₂;
Ac-Ile-Lys(Capryloyl)-Pqa-Arg-His-Tyr-Leu-Asn-Trp-
Val-Thr-Arg-Gln-(NMe)Arg-Tyr-NH₂;
Ac-Ile-Lys(Lauroyl)-Pqa-Arg-His-Tyr-Leu-Asn-Trp-
Val-Thr-Arg-Gln-(NMe)Arg-Tyr-NH₂;
H-Ile-Lys(Lauroyl-6-Ahx)-Pqa-Arg-His-Tyr-Leu-Asn-
Trp-Val-Thr-Arg-Gln-(NMe)Arg-Tyr-NH₂;
H-Ile-Lys(Lauroyl-beta-Ala)-Pqa-Arg-His-Tyr-Leu-
Asn-Trp-Val-Thr-Arg-Gln-(NMe)Arg-Tyr-NH₂;
H-Ile-Lys(Lauroyl-Glu)-Pqa-Arg-His-Tyr-Leu-Asn-
Trp-Val-Thr-Arg-Gln-(NMe)Arg-Tyr-NH₂;
H-Ile-Lys(Myristoyl-6-Ahx)-Pro-Pqa-Arg-His-Tyr-
Leu-Asn-Trp-Val-Thr-Arg-Gln-(NMe)Arg-Tyr-NH₂;
Ac-Ile-Lys(Palmitoyl)-Pqa-Arg-His-Tyr-Leu-Asn-Trp-
Val-Thr-Arg-Gln-(NMe)Arg-Tyr-NH₂;
H-Ile-Lys(Palmitoyl)-Pqa-Arg-His-Tyr-Leu-Asn-Trp-
Val-Thr-Arg-Gln-(NMe)Arg-Tyr-NH₂;
Palmitoyl-Ile-Lys-Pqa-Arg-His-Tyr-Leu-Asn-Trp-Val-
Thr-Arg-Gln-(NMe)Arg-T yr-NH₂;
Palmitoyl-6-Ahx-Ile-Lys-Pqa-Arg-His-Tyr-Leu-Asn-
Trp-Val-Thr-Arg-Gln-(NMe)Arg-Tyr-NH₂;
Palmitoyl-6-Ahx-Ile-Lys-Pqa-Arg-His-Tyr-Leu-Asn-
Trp-Val-Thr-Arg-Gln-Arg-Tyr-NH₂;
H-Ile-Lys(Palmitoyl-6-Ahx)-Pqa-Arg-His-Tyr-Leu-
Asn-Trp-Val-Thr-Arg-Gln-(NMe)Arg-Tyr-NH₂;
H-Ile-Lys(Palmitoyl-6-Ahx)-Pqa-Arg-His-Tyr-Leu-
Asn-Trp-Val-Thr-Arg-Gln-Arg-Tyr-NH₂;
H-Ile-Lys(Palmitoyl-beta-Ala)-Pqa-Arg-His-Tyr-Leu-
Asn-Trp-Val-Thr-Arg-Gln-(NMe)Arg-Tyr-NH₂;
H-Ile-Lys(Palmitoyl-Glu)-Pqa-Arg-His-Tyr-Leu-Asn-
Trp-Val-Thr-Arg-Gln-(NMe)Arg-Tyr-NH₂;
H-Ile-Lys(Palmitoyl-beta-Ala-Glu)-Pqa-Arg-His-Tyr-
Leu-Asn-Trp-Val-Thr-Arg-Gln-(NMe)Arg-Tyr-NH₂;
H-Ile-Lys(Palmitoyl-Glu-Glu-)-Pqa-Arg-His-Tyr-Leu-
Asn-Trp-Val-Thr-Arg-Gln-(NMe)Arg-Tyr-NH₂;
H-Ile-Lys(Palmitoyl-gamma-Glu)-Pqa-Arg-His-Tyr-
Leu-Asn-Trp-Val-Thr-Arg-Gln-(NMe)Arg-Tyr-NH₂;
and
H-Ile-Lys(Palmitoyl-gamma-Glu-gamma-Glu-)-Pqa-Arg-
His-Tyr-Leu-Asn-Trp-Val-Thr-Arg-Gln-(NMe)Arg-Tyr-
NH₂.

12. The neuropeptide-2 receptor agonist according to claim 1, selected from the group consisting of:
H-Ile-Lys(Palmitoyl-beta-Ala-gamma-Glu-)-Pqa-Arg-
His-Tyr-Leu-Asn-Trp-Val-Thr-Arg-Gln-(NMe)Arg-Tyr-
NH₂;
H-Ile-Lys(16-Bromohexadecanoyl-gamma-Glu-gamma-
Glu-)-Pqa-Arg-His-Tyr-Leu-Asn-Trp-Val-Thr-Arg-Gln-
(NMe)Arg-Tyr-NH₂;
Pyro-Glu-Ile-Lys(Palmitoyl-gamma-Glu-gamma-Glu-)-
Pqa-Arg-His-Tyr-Leu-Asn-Trp-Val-Thr-Arg-Gln-(NMe)
Arg-Tyr-NH₂
H-Ile-Lys(2-hexyldecanoyl-6-Ahx)-Pqa-Arg-His-Tyr-
Leu-Asn-Trp-Val-Thr-Arg-Gln-(NMe)Arg-Tyr-NH₂;
H-Ile-Lys(Licosanoyl-6-Ahx)-Pqa-Arg-His-Tyr-Leu-
Asn-Trp-Val-Thr-Arg-Gln-(NMe)Arg-Tyr-NH₂;
H-Ile-Lys(Licosanoyl-gamma-Glu-gamma-Glu-)-Pqa-
Arg-His-Tyr-Leu-Asn-Trp-Val-Thr-Arg-Gln-(NMe)Arg-
Tyr-NH₂;
H-Ile-Lys(Palmitoyl-15-ATOPA)-Pqa-Arg-His-Tyr-Leu-
Asn-Trp-Val-Thr-Arg-Gln-(NMe)Arg-Tyr-NH₂;
H-Ile-Lys(Licosanoyl-1 5-ATOPA)-Pqa-Arg-His-Tyr-
Leu-Asn-Trp-Val-Thr-Arg-Gln-(NMe)Arg-Tyr-NH₂;
H-Ile-Lys(Palmitoyl-1 2-ATODA)-Pqa-Arg-His-Tyr-
Leu-Asn-Trp-Val-Thr-Arg-Gln-(NMe)Arg-Tyr-NH₂;
H-Ile-Lys(Licosanoyl-1 2-ATODA)-Pqa-Arg-His-Tyr-
Leu-Asn-Trp-Val-Thr-Arg-Gln-(NMe)Arg-Tyr-NH₂;
H-Ile-Lys(Palmitoyl-8-ADOSA)-Pqa-Arg-His-Tyr-Leu-
Asn-Trp-Val-Thr-Arg-Gln-(NMe)Arg-Tyr-NH₂;
H-Ile-Lys(Licosanoyl-8-ADOSA)-Pqa-Arg-His-Tyr-Leu-
Asn-Trp-Val-Thr-Arg-Gln-(NMe)Arg-Tyr-NH₂;
H-Ile-Lys(Palmitoyl-5-AOPSA)-Pqa-Arg-His-Tyr-Leu-
Asn-Trp-Val-Thr-Arg-Gln-(NMe)Arg-Tyr-NH₂;
H-Ile-Lys(Licosanoyl-5-AOPSA)-Pqa-Arg-His-Tyr-Leu-
Asn-Trp-Val-Thr-Arg-Gln-(NMe)Arg-Tyr-NH₂;
H-Ile-Lys(Palmitoyl-Ser-Ser)-Pqa-Arg-His-Tyr-Leu-
Asn-Trp-Val-Thr-Arg-Gln-(NMe)Arg-Tyr-NH₂;
H-Ile-Lys(Licosanoyl-Ser-Ser)-Pqa-Arg-His-Tyr-Leu-
Asn-Trp-Val-Thr-Arg-Gln-(NMe)Arg-Tyr-NH₂;
H-Ile-Lys(Palmitoyl-Thr-Thr)-Pqa-Arg-His-Tyr-Leu-
Asn-Trp-Val-Thr-Arg-Gln-(NMe)Arg-Tyr-NH₂;
and
H-Ile-Lys(Licosanoyl-Thr-Thr)-Pqa-Arg-His-Tyr-Leu-
Asn-Trp-Val-Thr-Arg-Gln-(NMe)Arg-Tyr-NH₂.

13. A pharmaceutical composition, comprising a therapeutically effective amount of the neuropeptide-2 receptor agonist according to claim 1, or a salt thereof, and a pharmaceutically acceptable carrier.
